(19) United States
(12) Patent Application Publication
(10) Pub. No.: US 2024/0158451 A1
(43) Pub. Date: May 16, 2024

(54) BIOSYNTHESIS OF MOGROSIDES

(71) Applicant: Ginkgo Gioworks, Inc., Boston, MA (US)

(72) Inventors: Diveena Becker, Quincy, MA (US); Josef Bober, South Boston, MA (US); Justin Michael Gardin, Watertown, MA (US); Matthew McMahon, Melrose, MA (US)

(21) Appl. No.: 18/281,479

(22) PCT Filed: Mar. 11, 2022
(86) PCT No.: PCT/US2022/019977

§ 371 Date: Sep. 11, 2023

Related U.S. Application Data
(60) Provisional application No. 63/160,712, filed on Mar. 12, 2021.

(51) Int. Cl. C07K 14/415; C12N 9/02; C12N 9/14; C12N 9/90; C12N 15/52; C12P 19/56; C12P 33/20

(52) U.S. Cl. CPC C07K 14/415; C12N 9/0071; C12N 9/0083; C12N 9/14; C12N 9/90; C12N 15/52; C12P 19/56; C12P 33/20; C12Y 114/14001; C12Y 114/99007; C12Y 303/02003; C12Y 504/99007; C12Y 504/99033

(57) ABSTRACT

Described in this application are proteins and host cells involved in methods of producing mogrol precursors, mogrol, and/or mogrosides.

### CROSS-REFERENCE TO RELATED APPLICATIONS

This application claims the benefit under 35 U.S.C. § 119(e) of U.S. Provisional Application No. 63/160,712, filed Mar. 12, 2021, entitled “BIOSYNTHESIS OF MOGROSIDES,” the entire disclosure of which is hereby incorporated by reference in its entirety.

### FIELD OF THE INVENTION

The present disclosure relates to the production of mogrol precursors, mogrol and mogrosides in recombinant cells.

REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. The ASCII file, created on Mar. 11, 2022, is named G091970076WO00-SEQ-FL.TXT and is 686,922 bytes in size.

### BACKGROUND

Mogrosides are glycosides of cucurbitane derivatives. Highly sought after as sweeteners and sugar alternatives, mogrosides are naturally synthesized in the fruits of plants, including Siraitia grosvenorii (S. grosvenorii). Although anti-cancer, anti-oxidative, and anti-inflammatory properties have been ascribed to mogrosides, characterization of the exact proteins involved in mogroside biosynthesis is limited. Furthermore, mogroside extraction from fruit is labor-intensive and the structural complexity of mogrosides often hinders de novo chemical synthesis.

### SUMMARY

Aspects of the present disclosure provide host cells and methods useful for the production of mogrol and/or mogrosides. In some embodiments, the host cell comprises a heterologous polynucleotide encoding a cytochrome b5 (CB5), wherein the host cell is capable of producing more mogrol than a control host cell that does not comprise the heterologous polynucleotide, and wherein the CB5 comprises: the amino acid sequence YTGLSP (SEQ ID NO: 47); the amino acid sequence KPLLMAIKGQIYDVS (SEQ ID NO: 48); the amino acid sequence LQDWEYKFM (SEQ ID NO: 49); and/or the amino acid sequence X₁X₂X₃EX₄GX₅X₆X₇X₈X₉X₁₀D (SEQ ID NO: 53), wherein: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I.

In some embodiments, the CB5 comprises: the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃F X₂₄X₂₅X₂₆X₂₇X₂₈X₂₉X₃₀X₃₁ (SEQ ID NO: 50), wherein: X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G; and/or the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), wherein: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L.

In some embodiments, the CB5 comprises one or more of the following amino acid sequences: QVWETLKEAIVAYTGLSPATFFTVLALGLAVYYVISGFFGTSDYGSH (SEQ ID NO: 58) or ELYWKAMEQIAWYTGLSPTAFFTILASMIFVFQMVSSMFVSPEEFNK (SEQ ID NO: 59); PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61); LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL(SEQ ID NO: 63); and KPAEDGPSESQAD (SEQ ID NO: 64) or EHSENGHRNFEID (SEQ ID NO: 65).

In some embodiments, the CB5 comprises: the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and/or the amino acid sequence LQDWEYKFM (SEQ ID NO: 49) at residues corresponding to positions 148-156 in SEQ ID NO: 1

In some embodiments, the CB5 comprises the amino acid sequence X₁X₂X₃EX₄GX₅X₆X₇X₈X₉X₁₀D (SEQ ID NO: 53) at residues corresponding to positions 190-202 of SEQ ID NO: 1.

In some embodiments, the CB5 comprises: the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃F X₂₄X₂₅X₂₆X₂₇X₂₈X₂₉X₃₀X₃₁ (SEQ ID NO: 50) at residues corresponding to positions 4-50 of SEQ ID NO: 1; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51) at residues corresponding to positions 64-104 of SEQ ID NO: 1; and/or the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52) at residues corresponding to positions 123-165 of SEQ ID NO: 1.

In some embodiments, the CB5 comprises at most one histidine in one or more of the following regions: a region corresponding to positions 64-104 of SEQ ID NO: 1; a region corresponding to positions 105-122 of SEQ ID NO: 1; and/or a region corresponding to positions 123-165 of SEQ ID NO: 1.

In some embodiments, the CB5 comprises no histidine residues in: a region corresponding to positions 64-104 of SEQ ID NO: 1; a region corresponding to positions 105-122 of SEQ ID NO: 1; and/or a region corresponding to positions 123-165 of SEQ ID NO: 1.

In some embodiments, the CB5 comprises a sequence that is at least 90% identical to any one of SEQ ID NOs: 1-3 and 318 In some embodiments, the CB5 comprises the sequence of any one of SEQ ID NOs: 1-3 and 318.

In some embodiments, the heterologous polynucleotide comprises a sequence that is at least 90% identical to any one of SEQ ID NOs: 11-14, 22-24, 316-317, and 330-331. In some embodiments, the heterologous polynucleotide comprises the sequence of any one of SEQ ID NOs: 11-14, 22-24, 316-317, and 330-331.

Further aspects of the present disclosure relate to host cells that comprise a heterologous polynucleotide encoding a cytochrome b5 (CB5), wherein the CB5 comprises a sequence that is at least 90% identical to any one of SEQ ID NOs: 1-10 and 318 and wherein the host cell is capable of producing mogrol.

In some embodiments, the CB5 comprises the sequence of any one of SEQ ID NOs: 1-10 and 318.

Further aspects of the present disclosure provide host cells that comprise a heterologous polynucleotide encoding a cytochrome b5 (CB5), wherein the CB5 comprises a sequence that is at least 90% identical to any one of SEQ ID NOs: 1-4 and 318 and wherein the host cell is capable of producing more mogrol than a control host cell that does not comprise the heterologous polynucleotide.

Further aspects of the present disclosure provide host cells that comprise a heterologous polynucleotide encoding a cytochrome b5 (CB5), wherein the heterologous polynucleotide comprises a sequence that is at least 90% identical to any one of SEQ ID NOs: 11-24, 316-317, and 330-331, and wherein the host cell is capable of producing mogrol.

In some embodiments, the heterologous polynucleotide comprises the sequence of any one of SEQ ID NOs: 11-24, 316-317, and 330-331.

Further aspects of the present disclosure provide host cells that comprise a heterologous polynucleotide encoding a cytochrome b5 (CB5), wherein the CB5 comprises: the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and/or the amino acid sequence DQATQKHRSFGFVTFLEKED (SEQ ID NO: 57) and wherein the host cell is capable of producing more mogrol than a control host cell that does not comprise the heterologous polynucleotide.

In some embodiments, the CB5 comprises: the amino acid sequence ILRVSFRKYRKAIEQ (SEQ ID NO: 54) at residues corresponding to positions 23-37 of SEQ ID NO: 4; the amino acid sequence RAFRPSIRFKKSHSTVPT (SEQ ID NO: 55) at residues corresponding to positions 53-70 of SEQ ID NO: 4; the amino acid sequence KNTLYVGG (SEQ ID NO: 56) at residues corresponding to positions 168-175 of SEQ ID NO: 4; and/or the amino acid sequence DQATQKHRSFGFVTFLEKED (SEQ ID NO: 57) at residues corresponding to positions 203-222 of SEQ ID NO: 4.

In some embodiments, the CB5 comprises a sequence that is at least 90% identical to SEQ ID NO: 4.

In some embodiments, the CB5 comprises SEQ ID NO: 4.

In some embodiments, the heterologous polynucleotide comprises a sequence that is at least 90% identical to SEQ ID NO: 15.

In some embodiments, the heterologous polynucleotide comprises SEQ ID NO: 15.

In some embodiments, the host cell is capable of producing more than 13.5 mg/L mogrol.

In some embodiments, the host cell further comprises one or more heterologous polynucleotides encoding one or more of: a UDP-glycosyltransferases (UGT) enzyme, a cucurbitadienol synthase (CDS) enzyme, a C11 hydroxylase, a cytochrome P450 reductase, an epoxide hydrolase (EPH), a lanosterol synthase and a squalene epoxidase (SQE). In some embodiments, the UGT enzyme comprises a sequence that is at least 90% identical to SEQ ID NO: 121. In some embodiments, the CDS enzyme comprises a sequence that is at least 90% identical to any one of SEQ ID NOs: 226, SEQ ID NO: 235, and SEQ ID NO: 232. In some embodiments, the C11 hydroxylase comprises a sequence that is at least 90% identical to any one of SEQ ID NOs: 280-281, 305,315, and 324. In some embodiments, the cytochrome P450 reductase comprises a sequence that is at least 90% identical to any one of SEQ ID NOs: 282-283 and 306-307. In some embodiments, the EPH comprises a sequence that is at least 90% identical to any one of SEQ ID NOs: 284-292 and 309-310. In some embodiments, the SQE comprises a sequence that is at least 90% identical to any one of SEQ ID NOs: 293-295, 312, or 328.

In some embodiments, the lanosterol synthase comprises a sequence that is at least 90% identical to SEQ ID NO: 329 or 336. In some embodiments, the SQE comprises a sequence that is at least 90% identical to SEQ ID NO: 312 or 328.

In some embodiments, the host cell is a yeast cell, a plant cell, or a bacterial cell. In some embodiments, the host cell is a yeast cell. In some embodiments, the yeast cell is a Saccharomyces cerevisiae or Yarrowia lipolytica cell. In some embodiments, the host cell is a bacterial cell. In some embodiments, the bacterial cell is an E. coli cell.

Further aspects of the present disclosure relate to methods of producing mogrol comprising culturing any of the host cells of the disclosure.

Further aspects of the present disclosure relate to methods of producing a mogroside comprising culturing any of the host cells of the disclosure.

In some embodiments, the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MITI), siamenoside I, mogroside IV (MIV), mogroside IVa (MIVA), isomogroside IV, mogroside III-E (MIIIE), mogroside V (MV), and/or mogroside VI (MVI).

Further aspects of the disclosure relate to bioreactors for producing mogrol or mogrosides, wherein the bioreactor comprises any of the host cells of the disclosure.

Further aspects of the disclosure relate to non-naturally occurring polynucleotides comprising a sequence that is at least 90% identical to any one of SEQ ID NOs: 11-14, 22-24, 316-317, and 330-331. In some embodiments, the polynucleotide encodes a cytochrome b5 (CB5) comprising a sequence that is at least 90% identical to any one of SEQ ID NOs: 1-10 and 318.

Further aspects of the disclosure relate to expression vectors comprising any of the non-naturally occurring polynucleotides of the disclosure.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1D include schematic overviews of putative mogrol biosynthesis pathways. SQS indicates squalene synthase, EPD indicates epoxidase, P450 indicates C11 hydroxylase, EPH indicates epoxide hydrolase, and CDS indicates cucurbitadienol synthase. FIG. 1A and FIG. 1B show putative mogrol biosynthesis pathways. FIG. 1C shows non-limiting examples of primary UGT activity. FIG. 1D shows non-limiting examples of secondary UGT activity.

FIGS. 2A-2B include graphs depicting mogrol production by strains comprising candidate proteins involved in mogroside biosynthesis that were included in a library that was screened as described in Example 1. Parent strain 669889 is the control base strain without a candidate protein. FIG. 2A is a graph showing results for all strains in the screen. FIG. 2B is a graph showing mogrol production by strains comprising a cytochrome b5 (CB5).

FIGS. 3A-3B include graphs depicting mogrol production by Y. lipolytica strains. FIG. 3A is a graph depicting mogrol production by Y. lipolytica strains expressing a CB5 protein with a sequence corresponding to SEQ ID NO: 1 (strain 994375) or a truncated form of the same CB5 protein with a sequence corresponding to SEQ ID NO: 318 (strain 934903). Strain 974137 lacks any S. grosvenorii cytochrome b5 protein and was used as a negative control. FIG. 3B includes a graph depicting mogrol production by Y. lipolytica strains expressing a CB5 proteins with a sequence corresponding to SEQ ID NO: 1 (strain 1338488), SEQ ID NO: 2 (strain 1338489), SEQ ID NO: 3 (strain 1338490). Strain 1419596 lacks any S. grosvenorii cytochrome b5 protein and was used as a negative control.

### DETAILED DESCRIPTION

Mogrosides are widely used as natural sweeteners, for example in beverages. However, de novo synthesis and mogroside extraction from natural sources often involves high production costs and low yield. This disclosure provides host cells that are engineered to efficiently produce mogrol (or 11,24,25-trihydroxy cucurbitadienol), mogrosides, and precursors thereof. Methods include heterologous expression of cucurbitadienol synthase (CDS) enzymes, UDP-glycosyltransferase (UGT) enzymes, C11 hydroxylase enzymes, cytochrome P450 reductase enzymes, epoxide hydrolase (EPH) enzymes, squalene epoxidase (SQE) enzymes, or combinations thereof. Examples 1 and 2 describes the identification and functional characterization of proteins that increase mogrol production, including cytochrome b5 (CB5). Proteins and host cells described in this disclosure can be used for making mogrol, mogrosides, and precursors thereof.

### Synthesis of Mogrol and Mogrosides

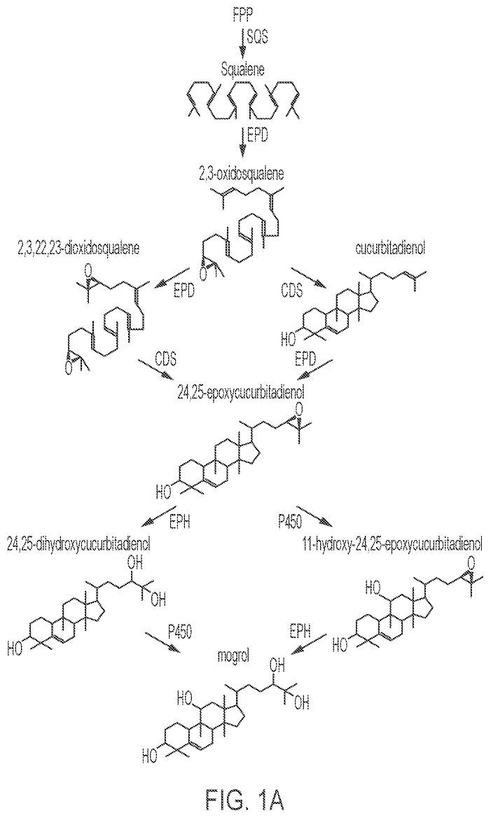

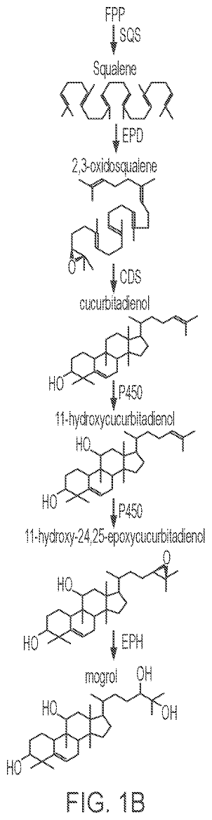

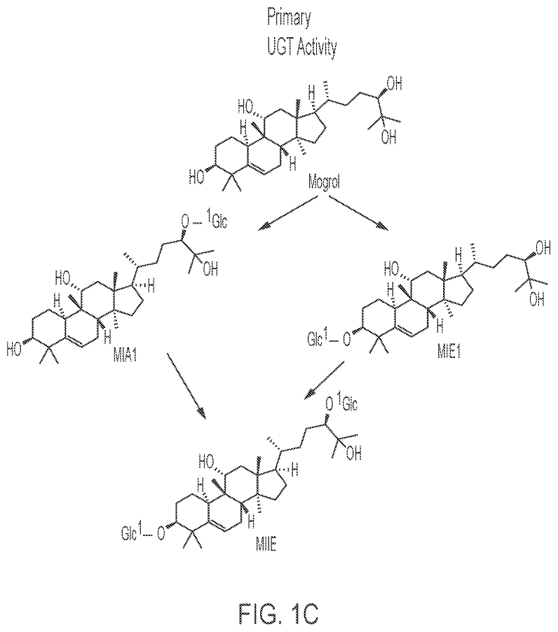

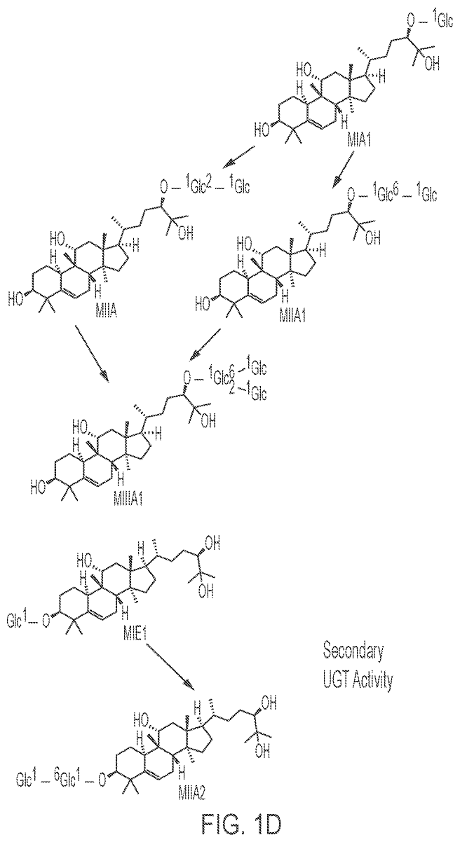

FIGS. 1A-1B show putative mogrol synthesis pathways. An early step in the pathway involves conversion of squalene to 2,3-oxidoqualene. As shown in FIG. 1A, 2,3-oxidosqualene can be first cyclized to cucurbitadienol followed by epoxidation to form 24,25-epoxycucurbitadienol, or 2,3-oxidosqualene can be epoxidized to 2,3,22,23-dioxidosqualene and then cyclized to 24,25-epoxycucurbitadienol. Next, the 24,25-epoxycucurbitadienol can be converted to mogrol (an aglycone of mogrosides) following epoxide hydrolysis and then oxidation, or oxidation and then epoxide hydrolysis. As shown in FIG. 1B, 2,3-oxidosqualene can be first cyclized to cucurbitadienol, which is then converted to 11-hydroxycucurbitadienol by a cytochrome P450 C11 hydroxylase. Then, a cytochrome P450 C11 hydroxylase may convert 11-hydroxycucurbitadienol to 11-hydroxy-24,25-epoxycucurbitadienol. 11-hydroxy-24,25-epoxycucurbitadienol may be converted to mogrol by epoxide hydrolase. C11 hydroxylases act in conjunction with cytochrome P450 reductases (not shown in FIGS. 1A-1B).

Mogrol can be distinguished from other cucurbitane triterpenoids by oxygenations at C3, C11, C24, and C25. Glycosylation of mogrol, for example at C3 and/or C24, leads to the formation of mogrosides.

Mogrol precursors include but are not limited to squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol. The term “dioxidosqualene” may be used to refer to 2,3,22,23-diepoxy squalene or 2,3,22,23-dioxido squalene. The term “2,3-epoxysqualene” may be used interchangeably with the term “2-3-oxidosqualene.” As used in this application, mogroside precursors include mogrol precursors, mogrol and mogrosides.

Examples of mogrosides include, but are not limited to, mogroside I-A1 (MIA1), mogroside IE (MIE or M1E), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), and mogroside VI (MVI or M6). In some embodiments, the mogroside produced is siamenoside I, which may be referred to as Siam. In some embodiments, the mogroside produced is MIIIE. Unless otherwise noted, when used in the plural, the terms “M1s”, “MIs”, “M2s”, “MIIs”, “M3s”, “MIIIs”, “M4s”, “MIVs”, “MVs”, “M5s”, “M6s”, and “MVIs” each refer to a class of mogrosides. As a non-limiting example, M2s or MIIs may include MIIA1, MIIA, MIIA2, and/or MIIE.

In other embodiments, a mogroside is a compound of Formula 1:

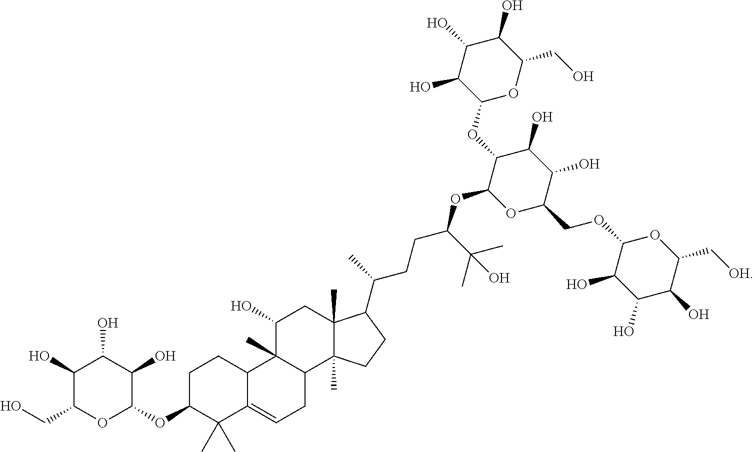 [SMILES: C[C@H](CC[C@@H](O[C@@H]1O[C@H](CO[C@@H]2O[C@H](CO)[C@@H](O)[C@H](O)[C@H]2O)[C@@H](O)[C@H](O)[C@H]1O[C@H]1O[C@@H](CO)[C@H](O)[C@@H](O)[C@@H]1O)C(C)(C)O)C1CC[C@@]2(C)C3CC=C4C(CC[C@H](O[C@@H]5O[C@H](CO)[C@@H](O)[C@H](O)[C@H]5O)C4(C)C)[C@]3(C)[C@H](O)C[C@]12C]

In some embodiments, the methods described in this application may be used to produce any of the compounds described in and incorporated by reference from US 2019/0071705 (which granted as U.S. Pat. No. 11,060,124), including compounds 1-20 as disclosed in US 2019/0071705. In some embodiments, the methods described in this application may be used to produce variants of any of the compounds described in and incorporated by reference from US 2019/0071705, including variants of compounds 1-20 as disclosed in US 2019/0071705. For example, a variant of a compound described in US 2019/0071705 can comprise a substitution of one or more alpha-glucosyl linkages in a compound described in US 2019/0071705 with one or more beta-glucosyl linkages. In some embodiments, a variant of a compound described in US 2019/0071705 comprises a substitution of one or more beta-glucosyl linkages in a compound described in US 2019/0071705 with one or more alpha-glucosyl linkages. In some embodiments, a variant of a compound described in US 2019/0071705 is a compound of Formula 1 shown above.

In some embodiments, a host cell comprising one or more proteins described herein (e.g., a cytochrome b5 (CB5), a UDP-glycosyltransferase (UGT) enzyme, a cucurbitadienol synthase (CDS) enzyme, a C11 hydroxylase enzyme, a cytochrome P450 reductase enzyme, an epoxide hydrolase enzyme (EPH), a squalene epoxidase enzyme (SQE) and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, or at least 10,000 mg/L of one or more mogrosides and/or mogroside precursors. In some embodiments, the mogroside is mogroside I-A1 (MIA1), mogroside IE (MIE or M1E), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), or mogroside VI (MVI or M6).

### Cytochrome b5 (CB5)

Aspects of the present disclosure provide cytochrome b5 (CB5) proteins, which may be useful in promoting mogrol production. As used herein, a “cytochrome b5” or “CB5” refers to a protein that comprises a lipid binding domain or cytochrome b5-like heme binding domain. In some embodiments, a lipid binding domain is a steroid binding domain.

CB5 proteins are heme- or lipid-binding proteins. For example, a CB5 may be a steroid binding protein. Some have been implicated in electron transport and enzymatic redox reactions. CB5 proteins generally harbor a conserved CB5 domain (e.g., a cytochrome b5-like heme or steroid binding domain). The tertiary structure of the CB5 domain is highly conserved and the domain folds around two hydrophobic residue cores on each side of a beta sheet. Without wishing to be bound by any theory, one hydrophobic core may include the heme or lipid binding domain, while the other hydrophobic core may promote formation of the proper conformation. In some embodiments, a lipid binding domain is a steroid binding domain.

Without being bound by a particular theory, two histidine residues may be required for a CB5 to interact with the iron in heme and CB5s that do not comprise these conserved histidine residues may comprise a lipid binding domain (e.g., a steroid binding domain) instead of a heme-binding domain. In some embodiments, a CB5 that is capable of increasing mogrol production does not comprise two histidine residues in a region corresponding to positions 64-104 of SEQ ID NO: 1, in a region corresponding to positions 105-122 of SEQ ID NO: 1, and/or in a region corresponding to positions 123-165 of SEQ ID NO: 1. In some embodiments, a CB5 that is capable of increasing mogrol production comprises at most one histidine in a region corresponding to positions 64-104 of SEQ ID NO: 1, in a region corresponding to positions 105-122 of SEQ ID NO: 1, and/or in a region corresponding to positions 123-165 of SEQ ID NO: 1.

In some embodiments, a CB5 that is capable of increasing mogrol production comprises no histidine residues in a region corresponding to positions 64-104 of SEQ ID NO: 1, in a region corresponding to positions 105-122 of SEQ ID NO: 1, and/or in a region corresponding to positions 123-165 of SEQ ID NO: 1.

A non-limiting example of a CB5 domain is provided under Pfam Accession No. PF00173. The CB5 domain may form a majority of the protein's structure. See e.g., SEQ ID NOs: 1-3 or 318. In some embodiments, additional domains such as a fatty acid desaturase and/or a FMN-dependent dehydrogenase are also present.

CB5 proteins may serve as an electron transfer component of a redox reaction. For example, a CB5 may function as an obligate electron donor in an oxidative reaction. In some embodiments, a CB5 serves as an electron-delivery partner for a cytochrome P450 (e.g., a C11 hydroxylase). In some embodiments, a CB5 catalyzes or promotes electron transfer from NADPH to a cytochrome P450 enzyme (e.g., a C11 hydroxylase).

In some embodiments, a CB5 plays an allosteric role to promote mogrol production. As a non-limiting example, a CB5 may be involved in binding and positioning of cucurbitadienol or cucurbitadienol-like molecules to support P450 enzyme activity. In some embodiments, a CB5 sterically interacts with the P450 enzyme to support an enzyme conformation that promotes higher activity, without a direct enzymatic role of the CB5 itself.

The rate of an enzymatic redox reaction may be assessed by any suitable method, including determination of the change in product concentration over a period of time. Any suitable method including mass spectrometry may be used to measure the presence of a substrate or product. See also, e.g., Schenkman et al., Pharmacology & Therapeutics 97 (2003) 139-152; Gou et al., Plant Cell. 2019 June; 31(6):1344-1366; Interpro Accession No. IPR001199; Interpro Accession No. IPR018506; Lederer Biochimie. 1994; 76(7):674-92; GenBank Accession No. AF332415; UniProt Accession No. P40312.

In some embodiments, a CB5 is 200-300 amino acids in length (e.g., 210-290 amino acids in length, 205-215 amino acids in length, or 275-295 amino acids in length).

In some embodiments, a CB5 of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 1-24, 316-318, 330-331, or any CB5 sequence disclosed in this application or known in the art. In some embodiments, a CB5 of the present disclosure comprises a sequence that is a conservatively substituted version of any one of SEQ ID NOs: 1-10 and 318.

In some embodiments, a CB5 comprises one or more motifs. As a non-limiting example, a motif may distinguish a CB5 that is capable of increasing mogrol production from a CB5 that does not increase mogrol production relative to a control.

In some embodiments, a CB5 comprises the amino acid sequence YTGLSP (SEQ ID NO: 47); the amino acid sequence KPLLMAIKGQIYDVS (SEQ ID NO: 48); and/or the amino acid sequence LQDWEYKFM (SEQ ID NO: 49). In some embodiments, the CB5 comprises the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and/or the amino acid sequence LQDWEYKFM (SEQ ID NO: 49) at residues corresponding to positions 148-156 in SEQ ID NO: 1. In some embodiments, a CB5 comprises the amino acid sequence YTGLSP (SEQ ID NO: 47); the amino acid sequence KPLLMAIKGQIYDVS (SEQ ID NO: 48); and/or the amino acid sequence LQDWEYKFM (SEQ ID NO: 49). In some embodiments, the CB5 comprises the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and the amino acid sequence LQDWEYKFM (SEQ ID NO: 49) at residues corresponding to positions 148-156 in SEQ ID NO: 1.

In some embodiments, a CB5 comprises the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃F X₂₄X₂₅X₂₆X₂₇X₂₈X₂₉X₃₀X₃₁ (SEQ ID NO: 50), in which X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H. As a non-limiting example, a CB5 comprising SEQ ID NO: 50 may comprise the amino acid sequence QVWETLKEAIVAYTGLSPATFFTVLALGLAVYYVISGFFGTSDYGSH (SEQ ID NO: 58) or the amino acid sequence ELYWKAMEQIAWYTGLSPTAFFTILASMIFVFQMVSSMFVSPEEFNK (SEQ ID NO: 59). In some embodiments, a CB5 may comprise SEQ ID NO: 50 at residues corresponding to positions 4-50 of SEQ ID NO: 1.

In some embodiments, a CB5 comprises the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G. As a non-limiting example, a CB5 comprising SEQ ID NO: 51 may comprise the amino acid sequence PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61). In some embodiments, a CB5 may comprise SEQ ID NO: 51 at residues corresponding to positions 64-104 of SEQ ID NO: 1.

In some embodiments, a CB5 comprises the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), in which: X₁ is the amino acid K or L; X₂ is the amino acid M or L; X₃ is the is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L. In some embodiments, a CB5 comprising SEQ ID NO: 52 may comprise LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63). In some embodiments, a CB5 comprises SEQ ID NO: 52 at residues corresponding to positions 123-165 of SEQ ID NO: 1.

In some embodiments, a CB5 comprises the amino acid sequence X₁X₂X₃EX₄GX₅X₆X₇X₈X₉X₁₀D (SEQ ID NO: 53), in which: X₁ is the amino acid K or E; X₂ is 10 the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I. In some embodiments, a CB5 comprising SEQ ID NO: 53 comprises KPAEDGPSESQAD (SEQ ID NO: 64) or EHSENGHRNFEID (SEQ ID NO: 65). In some embodiments, a CB5 comprises SEQ ID NO: 53 at residues corresponding to positions 190-202 of SEQ ID NO: 1.

In some embodiments, a CB5 comprises the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃F X₂₄X₂₅X₂₆X₂₇X₂₈X₂₉X₃₀X₃₁ (SEQ ID NO: 50), in which X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X_{2 7} is the amino acid E or D; X₂₈ is the amino acid E or Y; X₂₉ is the amino acid F or G; X₃₀ is the amino acid N or S; and/or X₃₁ is the amino acid K or H; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G; and the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X_{16 5} (SEQ ID NO: 52), in which: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L. In some embodiments, the CB5 further comprises the amino acid sequence X₁X₂X₃EX₄GX₅X₆X₇X₈X₉X₁₀D (SEQ ID NO: 53), in which: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I.

In some embodiments, a CB5 comprises the amino acid sequence QVWETLKEAIVAYTGLSPATFFTVLALGLAVYYVISGFFGTSDYGSH (SEQ ID NO: 58) or the amino acid sequence ELYWKAMEQIAWYTGLSPTAFFTILASMIFVFQMVSSMFVSPEEFNK (SEQ ID NO: 59); the amino acid sequence PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61); and the amino acid sequence LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63). In some embodiments, the CB5 further comprises KPAEDGPSESQAD (SEQ ID NO: 64) or EHSENGHRNFEID (SEQ ID NO: 65).

In some embodiments, a CB5 comprises the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and/or the amino acid sequence DQATQKHRSFGFVTFLEKED (SEQ ID NO: 57). In some embodiments, a CB5 comprises the amino acid sequence ILRVSFRKYRKAIEQ (SEQ ID NO: 54) at residues corresponding to positions 23-37 of SEQ ID NO: 4; the amino acid sequence RAFRPSIRFKKSHSTVPT (SEQ ID NO: 55) at residues corresponding to positions 53-70 of SEQ ID NO: 4; the amino acid sequence KNTLYVGG (SEQ ID NO: 56) at residues corresponding to positions 168-175 of SEQ ID NO: 4; and/or the amino acid sequence DQATQKHRSFGFVTFLEKED (SEQ ID NO: 57) at residues corresponding to positions 203-222 of SEQ ID NO: 4. In some embodiments, a CB5 comprises the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and the amino acid sequence DQATQKHRSFGFVTFLEKED (SEQ ID NO: 57).

In some embodiments, a CB5 is capable of increasing production of a mogrol precursor, mogrol, and/or a mogroside by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between relative to production of the mogrol precursor, mogrol, and/or the mogroside by a host cell that does not comprise the CB5. In some embodiments, a CB5 is capable of increasing production of a mogrol precursor, mogrol, and/or a mogroside by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between relative to production of the mogrol precursor, mogrol, and/or the mogroside by a host cell that does not comprise the CB5. In some embodiments, a CB5 is capable of increasing production of a mogrol precursor, mogrol, and/or a mogroside by a host cell between 0.01% and 1%, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between relative to production of the mogrol precursor, mogrol, and/or the mogroside by a host cell that does not comprise the CB5.

In some embodiments, a host cell comprising a CB5 is capable of producing at least 0.01 mg/L, at least 0.05 mg/L, at least 1 mg/L, at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 150 mg/L, at least 200 mg/L, at least 250 mg/L, at least 300 mg/L, at least 350 mg/L, at least 400 mg/L, at least 450 mg/L, at least 500 mg/L, at least 550 mg/L, at least 600 mg/L, at least 650 mg/L, at least 700 mg/L, at least 750 mg/L, at least 800 mg/L, at least 850 mg/L, at least 900 mg/L, at least 950 mg/L, or at least 1000 mg/L, including all values of a mogrol precursor, mogrol, and/or a mogroside. In some embodiments, a host cell comprising a CB5 is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, or at most 1000 mg/L of a mogrol precursor, mogrol, and/or mogroside. In some embodiments, a host cell comprising a CB5 is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, or between 1 mg/L and 1,000 mg/L, including all values in between of a mogrol precursor, mogrol, and/or the mogroside. As a non-limiting example, a CB5 may be capable of increasing production of a mogrol precursor, mogrol, and/or mogroside by a host cell that comprises one or more squalene synthases, epoxidases, cytochrome P450 reductases, C11 hydroxylases, epoxide hydrolases, and/or cucurbitadienol synthases. In some instances, a CB5 is capable of increasing production of a mogrol precursor, mogrol, and/or mogroside by a host cell that comprises one or more squalene synthases, epoxidases, cytochrome P450 reductases, C11 hydroxylases, epoxide hydrolases, cucurbitadienol synthases, and/or UDP-glycosyltransferases. In some embodiments, a host cell further comprises a CB5 reductase. In some embodiments, a host cell further comprises a glucanase.

### UDP-Glycosyltransferases (UGT) Enzymes

Aspects of the present disclosure provide UDP-glycosyltransferase enzymes (UGTs), which may be useful, for example, in the production of a mogroside (e.g., mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, or mogroside VI).

As used in this disclosure, a “UGT” refers to an enzyme that is capable of catalyzing the addition of the glycosyl group from a UTP-sugar to a compound (e.g., mogroside or mogrol). A UGT may be a primary and/or a secondary UGT.

A “primary” UGT, or a UGT that has “primary glycosylation activity,” refers to a UGT that is capable of catalyzing the addition of a glycosyl group to a position on a compound that does not comprise a glycosyl group. For example, a primary UGT may be capable of adding a glycosyl group to the C3 and/or C24 position of an isoprenoid substrate (e.g., mogrol). See, e.g., FIG. 1C.

A “secondary” UGT, or a UGT that has “secondary glycosylation activity,” refers to a UGT that is capable of catalyzing the addition of a glycosyl group to a position on a compound that already comprises a glycosyl group. See, e.g., FIG. 1D. As a non-limiting example, a secondary UGT may add a glycosyl group to a mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, and/or mogroside VI.

In some embodiments, a UGT (e.g., primary or secondary UGT) of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to any UGT sequence disclosed in this application or known in the art. In some embodiments, a UGT comprises a sequence that is a conservatively substituted version of SEQ ID NOs: 121.

The UGTs of the present disclosure may be capable of glycosylating mogrol or a mogroside at any of the oxygenated sites (e.g., at C3, C11, C24, and C25). In some embodiments, the UGT is capable of branching glycosylation (e.g., branching glycosylation of a mogroside at C3 or C24).

Non-limiting examples of suitable substrates for the UGTs of the present disclosure include mogrol and mogrosides (e.g., mogroside IA1 (MIA1), mogroside IE (MIE), mogroside II-A1 (MIIA1), mogroside III-A1 (MIIIA1), mogroside II-E (MIIE), mogro side III (MIII), or mogroside III-E (MIIIE), siamenoside I).

In some embodiments, the UGTs of the present disclosure are capable of producing mogroside IA1 (MIA1), mogroside IE (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, and/or mogroside V.

In some embodiments, the UGT is capable of catalyzing the conversion of mogrol to MIA1; mogrol to MIE1; MIA1 to MIIA1; MIE1 to MIIE; MIIA1 to MIIIA1; MIA1 to MIIE; MIIA1 to MIII; MIIIA1 to siamenoside I; MIIE to MIII; MIII to siamenoside I; MIIE to MIIE; and/or MIIIE to siamenoside I.

It should be appreciated that activity, such as specific activity, of a UGT can be measured by any means known to one of ordinary skill in the art. In some embodiments, the activity, such as specific activity, of a UGT may be determined by measuring the amount of glycosylated mogroside produced per unit enzyme per unit time. For example, the activity, such as specific activity, may be measured in mmol glycosylated mogroside target produced per gram of enzyme per hour. In some embodiments, a UGT of the present disclosure may have an activity, such as specific activity, of at least 0.1 mmol (e.g., at least 1 mmol, at least 1.5 mmol, at least 2 mmol, at least 2.5 mmol, at least 3, at least 3.5 mmol, at least 4 mmol, at least 4.5 mmol, at least 5 mmol, at least 10 mmol, including all values in between) glycosylated mogroside target produced per gram of enzyme per hour.

In some embodiments, the activity, such as specific activity, of a UGT of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control UGT. In some embodiments, the control UGT is a primary UGT. In some embodiments, the control UGT is a secondary UGT. In some embodiments, the control UGT is UGT94-289-1 (a wildtype UGT sequence from the monk fruit Siraitia grosvenorii provided by SEQ ID NO: 121). In some embodiments, for a UGT that has an amino acid substitution, a control UGT is the same UGT but without the amino acid substitution.

It should be appreciated that one of ordinary skill in the art would be able to characterize a protein as a UGT enzyme based on structural and/or functional information associated with the protein. For example, a protein can be characterized as a UGT enzyme based on its function, such as the ability to produce one or more mogrosides in the presence of a mogroside precursor, such as mogrol.

A UGT enzyme can be further characterized as a primary UGT based on its function of catalyzing the addition of a glycosyl group to a position on a compound that does not comprise a glycosyl group. A UGT enzyme can be characterized as a secondary UGT based on its function of catalyzing the addition of a glycosyl group to a position on a compound that already comprises a glycosyl group. In some embodiments, a UGT enzyme can be characterized as a both primary and a secondary UGT enzyme.

In other embodiments, a protein can be characterized as a UGT enzyme based on the percent identity between the protein and a known UGT enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the UGT sequences described in this application or the sequence of any other UGT enzyme.

In other embodiments, a protein can be characterized as a UGT enzyme based on the presence of one or more domains in the protein that are associated with UGT enzymes. For example, in certain embodiments, a protein is characterized as a UGT enzyme based on the presence of a sugar binding domain and/or a catalytic domain, characteristic of UGT enzymes known in the art. In certain embodiments, the catalytic domain binds the substrate to be glycosylated.

In other embodiments, a protein can be characterized as a UGT enzyme based on a comparison of the three-dimensional structure of the protein compared to the three-dimensional structure of a known UGT enzyme. For example, a protein could be characterized as a UGT based on the number or position of alpha helical domains, beta-sheet domains, etc. It should be appreciated that a UGT enzyme can be a synthetic protein.

Structurally, UGTs often comprise a UDPGT (Prosite: PS00375) domain and a catalytic dyad. As a non-limiting example, one of ordinary skill in the art may identify a catalytic dyad in a UGT by aligning the UGT sequence to UGT94-289-1 and identifying the two residues in the UGT that correspond to histidine 21 (H21) and aspartate 122 (D122) of UGT94-289-1.

The amino acid sequence for UGT94-289-1 is:
(SEQ ID NO: 121)
MDAQRGHTTTILMFPWLGYGHLSAFLELAKSLSRRNFHIYFCSTS
VNLDAIKPKLPSSSSSDSIQLVELCLPSSPDQLPPHLHTTNALPP
HLMPTLHQAFSMAAQHFAAILHTLAPHLLIYDSFQPWAPQLASSL
NIPAINFNTTGASVLTRMLHATHYPSSKFPISEFVLHDYWKAMYS
AAGGAVTKKDHKIGETLANCLHASCSVILINSFRELEEKYMDYLS
VLLNKKVVPVGPLVYEPNQDGEDEGYSSIKNWLDKKEPSSTVFVS
FGSEYFPSKEEMEEIAHGLEASEVHFIWVVRFPQGDNTSAIEDAL
PKGFLERVGERGMVVKGWAPQAKILKHWSTGGFVSHCGWNSVMES
MMFGVPIIGVPMHLDQPFNAGLAEEAGVGVEAKRDPDGKIQRDEV
AKLIKEVVVEKTREDVRKKAREMSEILRSKGEEKMDEMVAAISLF
LKI.
A non-limiting example of a nucleic acid
sequence encoding UGT94-289-1 is:
(SEQ ID NO: 325)
atggacgcgcaacgcggacatacgactaccatcctgatgtttccg
tggttggggtacggccaccttagtgcattcctcgaattagccaag
agcttgtcgcgtaggaactttcatatttatttctgttccacatct
gtcaatttagatgctataaaacccaaactaccatcatcttcaagt
tccgattctattcagcttgtagagttatgcttgccttcctcgcca
gaccaactacccccacacctgcatacaactaatgctctacctcca
catctaatgcctaccctgcaccaggccttttcaatggcagctcaa
cattttgcagctatattacatactttagcaccgcacttgttaatc
tatgattcgttccagccttgggcgccacaattggccagctctctt
aacattcctgctattaattttaataccacgggtgccagtgtgcta
acaagaatgttacacgcgactcattacccatcttcaaagttccca
atctccgaatttgttttacatgattattggaaagcaatgtattca
gcagctggtggtgctgttacaaaaaaggaccataaaataggagaa
accttggcaaactgtttacacgcttcttgctcggtaattctgatc
aattcattcagagagttggaagaaaaatacatggattacttgtct
gtcttactaaacaagaaagttgtgcccgtgggtccgcttgtttat
gagccaaaccaagatggcgaagacgaaggttatagttcgataaag
aattggctcgataaaaaggagccctcctcaactgtctttgtttcc
ttcgggtccgaatattttccgtccaaagaagaaatggaagaaatt
gcccatggcttggaggctagcgaggtacactttatttgggtcgtt
agattcccacaaggagacaatacttctgcaattgaagatgccctt
cctaagggttttcttgagcgagtgggcgaacgtggaatggtggtt
aagggttgggctcctcaggccaaaattttgaaacattggagcaca
ggcggtttcgtaagtcattgtggatggaatagtgttatggagagc
atgatgtttggtgtacccataataggtgttccgatgcatttagat
caaccatttaatgcagggctcgcggaagaagcaggagtaggggta
gaggctaaaagggaccctgatggtaagatacagagagatgaagtc
gctaaactgatcaaagaagtggttgtcgaaaaaacgcgcgaagat
gtcagaaagaaggctagggaaatgtctgaaattttacgttcgaaa
ggtgaggaaaagatggacgagatggttgcagccattagtctcttc
ttgaagatataa.

One of ordinary skill in the art would readily recognize how to determine for any UGT enzyme what amino acid residue corresponds to a specific amino acid residue in a reference UGT such as UGT94-289-1 (SEQ ID NO: 121) by, for example, aligning sequences and/or by comparing secondary or tertiary structures.

In certain embodiments, a UGT of the present disclosure comprises one or more structural motifs corresponding to a structural motif in wild-type UGT94-289-1 (e.g., corresponding to a structural motif that is shown in Table 1). In some embodiments, a UGT comprises structural motifs corresponding to all structural motifs in Table 1. In some embodiments, a UGT comprises a structural motif that corresponds to some but not all structural motifs shown in Table 1. In some embodiments, some structural motifs may diverge by having different lengths or different helicity. For example, a UGT of the present disclosure may comprise extended versions of loops 11, 16, 20, or a combination thereof. A UGT of the present disclosure may comprise loops that have greater helicity than their counterpart in UGT94-289-1 (e.g., loops 11, 16, 20, or a combination thereof in UGT94-289-1).

TABLE 1
Non-limiting Examples of Structural Motifs in
Reference Sequence UGT94-289-1 (SEQ ID NO: 121)

| | | | SEQ |
|---|---|---|---|
| Structural | | | ID |
| Motif | Borders | Sequence | NO |
| Loop 1 | Met1-Thr9 | MDAQRGHTT | 122 |
| Beta Sheet 1 | Thr10-Phe14 | TILMF | 123 |
| Loop 2 | Pro15-Gly18 | PWLG | 124 |
| Alpha Helix 1 | Tyr19-Arg34 | YGHLSAFLELA | 125 |
| | | KSLSR | |
| Loop3 | Arg35-Phe37 | RNF | 126 |
| Beta Sheet 2 | His38-Phe41 | HIYF | 127 |
| Loop 4 | Cys42-Thr44 | CST | 128 |
| Alpha Helix 2 | Ser45-Ala50 | SVNLDA | 129 |
| Loop 5 | Ile51-Ser61 | IKPKLPSSSSS | 130 |
| Beta Sheet 3 | Asp62-Gln65 | DSIQ | 131 |
| Loop 6 | Leu66-Leu88 | LVELCLPSSPDQLPP | 132 |
| | | HLHTTNAL | |
| Alpha Helix 3 | Pro89-Ala109 | PPHLMPTLHQAFSMA | 133 |
| | | AQHFAA | |
| Loop 7 | Ile110-His117 | ILHTLAPH | 134 |
| Beta Sheet 4 | Leu118-Asp122 | LLIYD | 135 |
| Loop 8 | Ser123-Pro126 | SFQP | 136 |
| Alpha Helix 4 | Trp127-Leu134 | WAPQLASSL | 137 |
| Loop 9 | Asn135-Pro137 | NIP | 138 |
| Beta Sheet 5 | Ala138-Asn143 | AINFN | 139 |
| Loop 10 | Thr144-Gly146 | TTG | 140 |
| Alpha Helix 5 | Ala147-His158 | ASVLTRMLHATH | 141 |
| Loop 11 | Tyr159-Tyr179 | YPSSKFPISEFV | 142 |
| | | LHDYWKAMY | |
| Alpha Helix 6 | Ser180-Gly183 | SAAG | 143 |
| Loop 12 | Gly184-Lys189 | GAVTKK | 144 |
| Alpha Helix 7 | Asp190-Ser204 | DHKIGETLANCL | 145 |
| | | HAS | |
| Loop 13 | Cys205-Ser206 | CS | 146 |
| Beta Sheet 6 | Val207-Ile210 | VILI | 147 |
| Loop 14 | Asn211-Glu217 | NSFRELE | 148 |
| Alpha Helix 8 | Glu218-Leu227 | EKYMDYLSVL | 149 |
| Loop 15 | Leu228-Asn229 | LN | 150 |
| Beta Sheet 7 | Lys230-Val232 | KKV | 151 |
| Loop 16 | Val233-Ser252 | VPVGPLVYEP | 152 |
| | | NQDGEDEGYS | |
| Alpha Helix 9 | Ser253-Lys261 | SIKNWLDKK | 153 |
| Loop 17 | Glu262-Ser265 | EPSS | 154 |
| Beta Sheet 8 | Thr266-Ser270 | TVFVS | 155 |
| Loop 18 | Phe271-Ser278 | FGSEYFPS | 156 |
| Alpha Helix 10 | Lys279-Ser292 | KEEMEEIAHGL | 157 |
| | | EAS | |
| Loop 19 | Glu293-His295 | EVH | 158 |
| Beta Sheet 9 | Phe296-Val300 | FIWVV | 159 |
| Alpha Helix 11 | Arg301-Asn307 | RFPQGDN | 160 |
| Loop 20 | Thr308-Gly318 | TSAIEDALPKG | 161 |
| Alpha Helix 12 | Phe319-Val323 | FLERV | 162 |
| Loop 21 | Gly324-Gly327 | GERG | 163 |
| Beta Sheet 10 | Met328-Lys331 | MVVK | 164 |
| Loop 22 | Gly332-Pro335 | GWAP | 165 |
| Alpha Helix 13 | Gln336-Lys341 | QAKILK | 166 |
| Loop 23 | His342-Gly346 | HWSTG | 167 |
| Beta Sheet 11 | Gly347-Ser350 | GFVS | 168 |
| Loop 24 | His351-Gly353 | HCG | 169 |
| Alpha Helix 14 | Trp354-Phe363 | WNSVMESMMF | 170 |
| Loop 25 | Gly364-Pro366 | GVP | 171 |
| Beta Sheet 12 | Ile367-Val370 | IIGV | 172 |
| Loop 26 | Pro371-Leu374 | PMHL | 173 |
| Alpha Helix 15 | Asp375-Ala386 | DQPFNAGLAEEA | 174 |
| Loop 27 | Gly387-Val388 | GV | 175 |
| Beta Sheet 13 | Gly389-Glu391 | GVE | 176 |
| Loop 28 | Ala392-Gln401 | AKRDPDGKIQ | 177 |
| Alpha Helix 16 | Arg402-Val414 | RDEVAKLIKEVVV | 178 |
| Loop 29 | Glu415 | E | 179 |
| Alpha Helix 17 | Lys416-Gly436 | KTREDVRKKAREMS | 180 |
| | | EILRSKG | |
| Loop 30 | Glu437-Met440 | EEKM | 181 |
| Alpha Helix 18 | Asp441-Leu451 | DEMVAAISLFL | 182 |
| Loop 31 | Lys452-Ile453 | KI | 183 |

In some embodiments, a UGT is a circularly permutated version of a reference UGT. In some embodiments, a UGT comprises a sequence that includes at least two motifs from Table 1 in a different order than a reference UGT. For example, if a reference UGT comprises a first motif that is located C-terminal to a second motif, the first motif may be located N-terminal to the second motif in a circularly permutated UGT.

A UGT may comprise one or more motifs selected from Loop 1, Beta Sheet 1, Loop 2, Alpha Helix 1, Loop 3, Beta Sheet 2, Loop 4, Alpha Helix 2, Loop 5, Beta Sheet 3, Loop 6, Alpha Helix 3, Loop 7, Beta Sheet 4, Loop 8, Alpha Helix 4, Loop 9, Beta Sheet 5, Loop 10, Alpha Helix 5, Loop 11, Alpha Helix 6, Loop 12, Alpha Helix 7, Loop 13, Beta Sheet 6, Loop 14, Alpha Helix 8, and Loop 15 from Table 1 located C-terminal to one or more motifs corresponding to one or more motifs selected from Beta Sheet 7, Loop 16, Alpha Helix 9, Loop 17, Beta Sheet 8, Loop 18, Alpha Helix 10, Loop 19, Beta Sheet 9, Alpha Helix 11, Loop 20, Alpha Helix 12, Loop 21, Beta Sheet 10, Loop 22, Alpha Helix 13, Loop 23, Beta Sheet 11, Loop 24, Alpha Helix 14, Loop 25, Beta Sheet 12, Loop 26, Alpha Helix 15, Loop 27, Beta Sheet 13, Loop 28, Alpha Helix 16, Loop 29, Alpha Helix 17, Loop 30, Alpha Helix 18, and Loop 31 in Table 1.

In some embodiments, the N-terminal portion of a UGT comprises a catalytic site, including a catalytic dyad, and/or a substrate-binding site. In some embodiments, the C-terminal portion of a UGT comprises a cofactor-binding site. Aspects of the disclosure include UGTs that have been circularly permutated. In some embodiments, in a circularly permutated version of a UGT, the N-terminal portion and the C-terminal portions may be reversed in whole or in part. For example, the C-terminal portion of a circularly permutated UGT may comprise a catalytic site, including a catalytic dyad, and/or a substrate-binding site, while the N-terminal portion may comprise a cofactor-binding site. In some embodiments, a circularly permutated version of a UGT comprises a heterologous polynucleotide encoding a UGT, wherein the UGT comprises: a catalytic dyad and a cofactor binding site, wherein the catalytic dyad is located C-terminal to the cofactor-binding site.

A circularly permutated UGT encompassed by the disclosure may exhibit different properties from the same UGT that has not undergone circular permutation. In some embodiments, a host cell expressing such a circularly permutated version of a UGT produces in the presence of at least one mogroside precursor at least 10%, 20%, 30%, 40%, 50%, 60%, 70%, 80%, 90%, or 100% more of one or more mogrosides relative to a host cell that comprises a heterologous polynucleotide encoding a reference UGT that is not circularly permutated, such as wild-type UGT94-289-1 (SEQ ID NO: 121). In some embodiments, a host cell expressing such a circularly permutated version of a UGT produces in the presence of at least one mogroside precursor at least 10%, 20%, 30%, 40%, 50%, 60%, 70%, 80%, 90%, or 100% less of one or more mogrosides relative to a host cell that comprises a heterologous polynucleotide encoding a reference UGT that is not circularly permutated, such as wild-type UGT94-289-1 (SEQ ID NO: 121).

### Cucurbitadienol Synthase (CDS) Enzymes

Aspects of the present disclosure provide cucurbitadienol synthase (CDS) enzymes, which may be useful, for example, in the production of a cucurbitadienol compound, such as 24-25 epoxy-cucurbitadienol or cucurbitadienol. CDSs are capable of catalyzing the formation of cucurbitadienol compounds, such as 24-25 epoxy-cucurbitadienol or cucurbitadienol from oxidosqualene (e.g., 2-3 -oxidosqualene or 2,3; 22,23 -diepoxysqualene).

In some embodiments, CDSs have a leucine at a residue corresponding to position 123 of SEQ ID NO: 256 that distinguishes them from other oxidosqualene cyclases, as discussed in Takase et al.Org. Biomol. Chem., 2015, 13, 7331-7336, which is incorporated by reference in its entirety.

CDSs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical, including all values in between, to a nucleic acid or amino acid sequence in Table 6, to a sequence selected from SEQ ID NO: 184-263, 299, 308, or 319, or to any other CDS sequence disclosed in this application or known in the art. In some embodiments, a CDS comprises a sequence that is a conservatively substituted version of any one of SEQ ID NOs: 224-263 or 308.

In some embodiments a CDS enzyme corresponds to AquAgaCDS16 (SEQ ID NO: 226), CSPIO6G07180.1 (SEQ ID NO: 235), or A0A1S3CBF6 (SEQ ID NO: 232).

In some embodiments, a nucleic acid sequence encoding a CDS enzyme may be codon optimized for expression in a particular host cell, including S. cerevisiae. In some embodiments, a codon-optimized nucleic acid sequence encoding a CDS enzyme corresponds to SEQ ID NO: 186, 195 or 192.

In some embodiments, a CDS of the present disclosure is capable of using oxidosqualene (e.g., 2,3-oxidosqualene or 2,3; 22,23-diepoxysqualene) as a substrate. In some embodiments, a CDS of the present disclosure is capable of producing cucurbitadienol compounds (e.g., 24-25 epoxy-cucurbitadienol or cucurbitadienol). In some embodiments, a CDS of the present disclosure catalyzes the formation of cucurbitadienol compounds (e.g., 24-25 epoxy-cucurbitadienol or cucurbitadienol) from oxidosqualene (e.g., 2-3-oxidosqualene or 2,3; 22,23-diepoxysqualene).

It should be appreciated that activity of a CDS can be measured by any means known to one of ordinary skill in the art. In some embodiments, the activity of a CDS may be measured as the normalized peak area of cucurbitadienol produced. In some embodiments, this activity is measured in arbitrary units. In some embodiments, the activity, such as specific activity, of a CDS of the present disclosure is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control CDS.

It should be appreciated that one of ordinary skill in the art would be able to characterize a protein as a CDS enzyme based on structural and/or functional information associated with the protein. For example, in some embodiments, a protein can be characterized as a CDS enzyme based on its function, such as the ability to produce cucurbitadienol compounds (e.g., 24-25 epoxy-cucurbitadienol or cucurbitadienol) using oxidosqualene (e.g., 2,3-oxidosqualene or 2,3; 22,23-diepoxysqualene) as a substrate. In some embodiments, a protein can be characterized, at least in part, as a CDS enzyme based on the presence of a leucine residue at a position corresponding to position 123 of SEQ ID NO: 256.

In some embodiments, a host cell that comprises a heterologous polynucleotide encoding a CDS enzyme produces at least 10%, 20%, 30%, 40%, 50%, 60%, 70%, 80%, 90%, or 100% more cucurbitadienol compound relative to the same host cell that does not express the heterologous gene.

In other embodiments, a protein can be characterized as a CDS enzyme based on the percent identity between the protein and a known CDS enzyme. For example, the protein may be at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical, including all values in between, to any of the CDS sequences described in this application or the sequence of any other CDS enzyme. In other embodiments, a protein can be characterized as a CDS enzyme based on the presence of one or more domains in the protein that are associated with CDS enzymes. For example, in certain embodiments, a protein is characterized as a CDS enzyme based on the presence of a substrate channel and/or an active-site cavity characteristic of CDS enzymes known in the art. In some embodiments, the active site cavity comprises a residue that acts a gate to this channel, helping to exclude water from the cavity. In some embodiments, the active-site comprises a residue that acts a proton donor to open the epoxide of the substrate and catalyze the cyclization process.

In other embodiments, a protein can be characterized as a CDS enzyme based on a comparison of the three-dimensional structure of the protein compared to the three-dimensional structure of a known CDS enzyme. It should be appreciated that a CDS enzyme can be a synthetic protein.

### C11 Hydroxylase Enzymes

Aspects of the present disclosure provide C11 hydroxylase enzymes, which may be useful, for example, in the production of mogrol.

A C11 hydroxylase of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a C11 hydroxylase sequence (e.g., nucleic acid or amino acid sequence) in Tables 7 and 8, with a sequence set forth as SEQ ID NO: 264-265, 280-281, 296, 305, 314-315, 320, 321, 324, 334, or 335 or to any C11 hydroxylase sequence disclosed in this application or known in the art. In some embodiments, a C11 hydroxylase comprises a sequence that is a conservatively substituted version of any one of SEQ ID NOs: 280-281, 305, 315, and 324.

In some embodiments, a C11 hydroxylase of the present disclosure is capable of oxidizing mogrol precursors (e.g., cucurbitadienol, 11-hydroxycucurbitadienol, 24,25-dihydroxy-cucurbitadienol, and/or 24,25-epoxy-cucurbitadienol). In some embodiments, a C11 hydroxylase of the present disclosure catalyzes the formation of mogrol.

It should be appreciated that activity, such as specific activity, of a C11 hydroxylase can be determined by any means known to one of ordinary skill in the art. In some embodiments, activity (e.g., specific activity) of a C11 hydroxylase may be measured as the concentration of a mogrol precursor produced or mogrol produced per unit of enzyme per unit time. In some embodiments, a C11 hydroxylase of the present disclosure has an activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity, such as specific activity, of a C11 hydroxylase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control C11 hydroxylase.

### Cytochrome P450 Reductase Enzymes

Aspects of the present disclosure provide cytochrome P450 reductase enzymes, which may be useful, for example, in the production of mogrol. Cytochrome P450 reductase is also referred to as NADPH:ferrihemoprotein oxidoreductase, NADPH:hemoprotein oxidoreductase, NADPH:P450 oxidoreductase, P450 reductase, POR, CPR, and CYPOR. These reductases can promote C11 hydroxylase activity by catalyzing electron transfer from NADPH to a C11 hydroxylase.

Cytochrome P450 reductases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a cytochrome P450 reductase sequence (e.g., nucleic acid or amino acid sequence) in Tables 7 and 8, with a sequence set forth as SEQ ID NO: 266-267, 282-283, 297-298, 306-307, 323, or 333 or to any cytochrome p450 reductase disclosed in this application or known in the art. In some embodiments, a cytochrome P450 reductase comprises a sequence that is a conservatively substituted version of any one of SEQ ID NOs: 282-283 and 306-307.

In some embodiments, a cytochrome P450 reductase of the present disclosure is capable of promoting oxidation of a mogrol precursor (e.g., cucurbitadienol, 11-hydroxycucurbitadienol, 24,25-dihydroxy-cucurbitadienol, and/or 24,25-epoxy-cucurbitadienol). In some embodiments, a P450 reductase of the present disclosure catalyzes the formation of a mogrol precursor or mogrol.

It should be appreciated that activity (e.g., specific activity) of a cytochrome P450 reductase can be measured by any means known to one of ordinary skill in the art. In some embodiments, activity (e.g., specific activity) of a recombinant cytochrome P450 reductase may be measured as the concentration of a mogrol precursor produced or mogrol produced per unit enzyme per unit time in the presence of a C11 hydroxylase. In some embodiments, a cytochrome P450 reductase of the present disclosure has a activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity (e.g., specific activity) of a cytochrome P450 reductase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control cytochrome P450 reductase.

### Epoxide Hydrolase Enzymes (EPHs)

Aspects of the present disclosure provide epoxide hydrolase enzymes (EPHs), which may be useful, for example, in the conversion of 24-25 epoxy-cucurbitadienol to 24-25 dihydroxy-cucurbitadienol or in the conversion of 11-hydroxy-24,25-epoxycucurbitadienol to mogrol. EPHs are capable of converting an epoxide into two hydroxyls.

EPHs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a EPH sequence (e.g., nucleic acid or amino acid sequence) in Tables 7 and 8, with a sequence set forth as SEQ ID NO: 268-276, 284-292, 300-301, 309-310, or 322, or to any EPH sequence disclosed in this application or known in the art. In some embodiments, an EPH comprises a sequence that is a conservatively substituted version of any one of SEQ ID NOs: 284-292 and 309-310.

In some embodiments, a recombinant EPH of the present disclosure is capable of promoting hydrolysis of an epoxide in a cucurbitadienol compound (e.g., hydrolysis of the epoxide in 24-25 epoxy-cucurbitadienol). In some embodiments, an EPH of the present disclosure catalyzes the formation of a mogrol precursor (e.g., 24-25 dihydroxy-cucurbitadienol).

It should be appreciated that activity (e.g., specific activity) of an EPH can be measured by any means known to one of ordinary skill in the art. In some embodiments, activity (e.g., specific activity) of an EPH may be measured as the concentration of a mogrol precursor (e.g., 24-25 dihydroxy-cucurbitadienol) or mogrol produced. In some embodiments, a recombinant EPH of the present disclosure will allow production of at least 1-100 μg/L, at least 100-1000 μg/L, at least 1-100 mg/L, at least 100-1000 mg/L, at least 1-10 g/L or at least 10-100 g/L, including all values in between.

In some embodiments, the activity (e.g., specific activity) of an EPH is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control EPH.

### Squalene Epoxidases Enzymes (SQEs)

Aspects of the present disclosure provide squalene epoxidases (SQEs), which are capable of oxidizing a squalene (e.g., squalene or 2-3-oxidosqualene) to produce a squalene epoxide (e.g., 2-3-oxidosqualene or 2-3, 22-23-diepoxysqualene). SQEs may also be referred to as squalene monooxygenases.

SQEs of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a SQE sequence (e.g., nucleic acid or amino acid sequence) in Tables 7 and 8, with a sequence set forth as SEQ ID NO: 277-279, 293-295, 303,312, 326, or 328, or to any SQE sequence disclosed in this application or known in the art.

In some embodiments, an SQE comprises a sequence that is a conservatively substituted version of any one of SEQ ID NOs: 293-295, 312, or 328.

In some embodiments, an SQE of the present disclosure is capable of promoting formation of an epoxide in a squalene compound (e.g., epoxidation of squalene or 2,3-oxidosqualene). In some embodiments, an SQE of the present disclosure catalyzes the formation of a mogrol precursor (e.g., 2-3-oxidosqualene or 2-3, 22-23-diepoxysqualene).

Activity, such as specific activity, of a recombinant SQE may be measured as the concentration of a mogrol precursor (e.g., 2-3-oxidosqualene or 2-3, 22-23-diepoxysqualene) produced per unit of enzyme per unit of time. In some embodiments, an SQE of the present disclosure has an activity, such as specific activity, of at least 0.0000001 μmol/min/mg (e.g., at least 0.000001 μmol/min/mg, at least 0.00001 μmol/min/mg, at least 0.0001 μmol/min/mg, at least 0.001 μmol/min/mg, at least 0.01 μmol/min/mg, at least 0.1 μmol/min/mg, at least 1 μmol/min/mg, at least 10 μmol/min/mg, or at least 100 μmol/min/mg, including all values in between).

In some embodiments, the activity, such as specific activity, of a SQE is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, or at least 100 fold, including all values in between) greater than that of a control SQE.

### Variants

Aspects of the disclosure relate to polynucleotides encoding any of the recombinant polypeptides described, such as CB5, CDS, UGT, C11 hydroxylase, cytochrome P450 reductase, EPH, SQE, and lanosterol synthase enzymes and any proteins associated with the disclosure. Variants of polynucleotide or amino acid sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” as known in the art, refers to a relationship between the sequences of two polypeptides or polynucleotides, as determined by sequence comparison (alignment). In some embodiments, sequence identity is determined across the entire length of a sequence, while in other embodiments, sequence identity is determined over a region of a sequence.

Identity can also refer to the degree of sequence relatedness between two sequences as determined by the number of matches between strings of two or more residues (e.g., nucleic acid or amino acid residues). Identity measures the percent of identical matches between the smaller of two or more sequences with gap alignments (if any) addressed by a particular mathematical model, algorithms, or computer program.

Identity of related polypeptides or nucleic acid sequences can be readily calculated by any of the methods known to one of ordinary skill in the art. The “percent identity” of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the protein molecules of the invention. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T.F. & Waterman, M.S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., CB5, CDS, UGT, C11 hydroxylase, cytochrome P450 reductase, EPH, or SQE variant or variant of any protein associated with the disclosure) comprises a domain that shares a secondary structure (e.g., alpha helix, beta sheet) with a reference polypeptide (e.g., a reference CB5, CDS, UGT, C11 hydroxylase, cytochrome P450 reductase, EPH, SQE, or any protein associated with the disclosure). In some embodiments, a polypeptide variant (e.g., CB5, CDS, UGT, C11 hydroxylase, cytochrome P450 reductase, EPH, or SQE variant or variant of any protein associated with the disclosure) shares a tertiary structure with a reference polypeptide (e.g., a reference CB5, CDS, UGT, C11 hydroxylase, cytochrome P450 reductase, EPH, SQE, or any protein associated with the disclosure). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

Mutations can be made in a nucleotide sequence by a variety of methods known to one of ordinary skill in the art. For example, mutations can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by gene editing tools, or by insertions, such as insertion of a tag (e.g., a HIS tag or a GFP tag). Mutations can include, for example, substitutions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a protein with different functional characteristics (e.g., increased or decreased enzymatic activity, different substrate specificity, or different product specificity). See, e.g., Yu and Lutz, Trends Biotechnol. 2011 Jan;29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the recombinant CB5s, CDSs, UGTs, C11 hydroxylases, cytochrome P450 reductases, EPHs, squalene epoxidases, and any other proteins disclosed in this application are also encompassed by the present disclosure. For example, functional variants may bind one or more of the same substrates (e.g., mogrol, mogroside, or precursors thereof) or produce one or more of the same products (e.g., mogrol, mogroside, or precursors thereof). Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain. For example, among oxidosqualene cyclases, additional CDS enzymes may be identified in some instances by searching for polypeptides with a leucine residue corresponding to position 123 of SEQ ID NO: 256. This leucine residue has been implicated in determining the product specificity of the CDS enzyme; mutation of this residue can, for instance, result in cycloartenol or parkeol as a product (Takase et al., Org Biomol Chem. 2015 Jul. 13(26):7331-6).

Additional UGT enzymes may be identified, for example, by searching for polypeptides with a UDPGT domain (PROSITE accession number PS00375).

Homology modeling may also be used to identify amino acid residues that are amenable to mutation without affecting function. A non-limiting example of such a method may include use of position-specific scoring matrix (PSSM) and an energy minimization protocol. See, e.g., Stormo et al., Nucleic Acids Res. 1982 May 11; 10(9):2997-3011.

PSSM may be paired with calculation of a Rosetta energy function, which determines the difference between the wild-type and the single-point mutant. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. doi: 10.1016/j.molcel.2016.06.012.

In some embodiments, a CB5, CDS, UGT, C11 hydroxylase, cytochrome P450 reductase, EPH, or SQE coding sequence or coding sequence of any protein associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions corresponding to a reference coding sequence. In some embodiments, the CB5, CDS, UGT, C11 hydroxylase, cytochrome P450 reductase, EPH, or SQE coding sequence or coding sequence of any protein associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a recombinant CB5, CDS, UGT, C11 hydroxylase, cytochrome P450 reductase, EPH, or SQE sequence or other recombinant protein sequence associated with the disclosure alter the amino acid sequence of the polypeptide relative to the amino acid sequence of a reference polypeptide. In some embodiments, the one or more mutations alter the amino acid sequence of the recombinant polypeptide relative to the amino acid sequence of a reference polypeptide and alter (enhance or reduce) an activity of the polypeptide relative to the reference polypeptide.

The activity, including specific activity, of any of the recombinant polypeptides described in this application may be measured using methods known in the art. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide coding sequence may result in conservative amino acid substitutions to provide functionally equivalent variants of the foregoing polypeptides, e.g., variants that retain the activities of the polypeptides. As used in this application, a “conservative amino acid substitution” or “conservatively substituted” refers to an amino acid substitution that does not alter the relative charge or size characteristics or functional activity of the protein in which the amino acid substitution is made.

In some instances, an amino acid is characterized by its R group (see, e.g., Table 2). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Additional non-limiting examples of conservative amino acid substitutions are provided in Table 2.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 2
Non-limiting examples of conservative amino acid substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asp, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide variant having a desired property and/or activity can be made by alteration of the coding sequence of the polypeptide. Similarly, conservative amino acid substitutions in the amino acid sequence of a polypeptide to produce functionally equivalent variants of the polypeptide typically are made by alteration of the coding sequence of the recombinant polypeptide (e.g., CB5, UGT, CDS, P450, cytochrome P450 reductase, EPH, squalene epoxidase, or any protein associated with the disclosure).

### Expression of Nucleic Acids in Host Cells

Aspects of the present disclosure relate to the recombinant expression of genes encoding proteins, functional modifications and variants thereof, as well as uses relating thereto. For example, the methods described in this application may be used to produce mogrol precursors, mogrol, and/or mogrosides.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system; or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is: situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez et al., Nat Methods. 2016 July; 13(7): 563-567. A heterologous polynucleotide may comprise a wild-type sequence or a mutant sequence as compared with a reference polynucleotide sequence.

A nucleic acid encoding any of the recombinant polypeptides, such as CB5s, CDSs, UGTs, C11 hydroxylases, cytochrome P450 reductases, EPHs, SQEs, or any proteins associated with the disclosure, described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

In some embodiments, a vector replicates autonomously in the cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell, such as a yeast cell. In some embodiments, the nucleic acid sequence of a gene described in this application is inserted into a cloning vector such that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, the nucleic acid sequence of a gene described in this application is codon-optimized. Codon optimization may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined or linked if induction of a promoter in the 5′ regulatory sequence permits the coding sequence to be transcribed and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1,TPI1 GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls icon, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, tetracycline, galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1,TPI1, HXT3, HXT7, ACT1, ADH1, ADH2, ENO2, and SOD1.

Other inducible promoters or constitutive promoters known to one of ordinary skill in the art are also contemplated.

Regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. Vectors may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a host cell is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

In some embodiments, introduction of a polynucleotide, such as a polynucleotide encoding a recombinant polypeptide, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the recombinant polypeptides described in this application, in its genome.

### Host Cells

Any of the proteins of the disclosure may be expressed in a host cell. As used in this application, the term “host cell” refers to a cell that can be used to express a polynucleotide, such as a polynucleotide that encodes a protein used in production of mogrol, mogrosides, and precursors thereof.

Any suitable host cell may be used to produce any of the recombinant polypeptides, including CB5s, CDSs, UGTs, C11 hydroxylases, cytochrome P450 reductases, EPHs, SQEs, and other proteins disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to, Candida, Escherichia, Hansenula, Saccharomyces (e.g., S. cerevisiae), Schizosaccharomyces, Pichia, Kluyveromyces (e.g., K. lactis), and Yarrowia (e.g., Y. lipolytica). In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Komagataella phaffii, Komagataella pastoris, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. Reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C. saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. carotovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell is an industrial Zymomonas species (e.g., Z mobilis, Z. lipolytica).

The present disclosure is also suitable for use with a variety of animal cell types, including mammalian cells, for example, human (including 293, HeLa, WI38, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae or Y. lipolytica) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-CoA) synthase (ERG13), farnesyl-diphosphate farnesyl transferase (squalene synthase) (ERGS), may be modified to overexpress squalene epoxidase (ERG1), or may be modified to downregulate lanosterol synthase (ERG7). In some embodiments, a host cell is modified to reduce or eliminate expression of one or more transporter genes, such as PDR1 or PDR3, and/or the glucanase gene EXG1.

In some embodiments, a host cell is modified to reduce or inactivate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes.

In some embodiments, a host cell is modified to reduce or inactivate 1, 2, 3, 4, 5, 6, 7, 8, 9, 10, 11, 12, 13, 14, 15, 16, 17, 18, 19, or 20 genes.

Reduction of gene expression and/or gene inactivation may be achieved through any suitable method, including but not limited to deletion of the gene, introduction of a point mutation into the gene, truncation of the gene, introduction of an insertion into the gene, introduction of a tag or fusion into the gene, or selective editing of the gene. For example, polymerase chain reaction (PCR)-based methods may be used (see, e.g., Gardner et al., Methods Mol Biol. 2014; 1205:45-78) or well-known gene-editing techniques may be used. As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104).

A vector encoding any of the recombinant polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks, Roux bottles, multiple-surface tissue culture propagators, modified fermenters, and coated beads (e.g., beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., mogrol precursor, mogrol, mogroside precursor, or mogroside) may display some differences from the substrate (e.g., mogrol precursor, mogrol, mogroside precursor, or mogroside) in terms of solubility, toxicity, cellular accumulation and secretion and in some embodiments can have different fermentation kinetics.

The methods described in this application encompass production of the mogrol precursors (e.g., squalene, 2,3-oxidosqualene, or 24-25 epoxy-cucurbitadienol), mogrol, or mogrosides (e.g., MIA1, MIE1, MIIA1, MIIA2, MIIIA1, MIIE, MIII, siamenoside I, mogroside IV, isomogroside IV, MIIIE, and mogroside V) using a recombinant cell, cell lysate or isolated recombinant polypeptides (e.g., CB5, CDS, UGT, C11 hydroxylase, cytochrome P450 reductase, EPH, squalene epoxidase, and any proteins associated with the disclosure).

Mogrol precursors (e.g., squalene, 2,3-oxidosqualene, or 24-25 epoxy-cucurbitadienol), mogrol, mogrosides (e.g., MIA1, MIE, MIIA1, MIIA2, MIIIA1, MIIE, MIII, siamenoside I, mogroside IV, isomogroside IV, MIIIE, and mogroside V) produced by any of the recombinant cells disclosed in this application may be identified and extracted using any method known in the art. Mass spectrometry (e.g., LC-MS, GC-MS) is a non-limiting example of a method for identification and may be used to help extract a compound of interest.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification and Functional Characterization of CB5 Proteins That Increase Mogrol Production

This Example describes the screening of S. grosvenorii proteins in S. cerevisiae to identify proteins that promote mogrol production. The library included approximately 333 S. grosvenorii proteins whose expression correlated with expression and/or matched enzyme class of proteins involved in mogroside biosynthesis. Transcriptomic data from Xia et al. Gigascience. 2018 Jun. 1; 7(6):giy067 was used for the analysis. The entire library was screened for mogrol production to determine whether proteins whose expression correlated with expression and/or matched enzyme class of one or more proteins involved in mogroside biosynthesis could be used to increase mogrol production.

S. cerevisiae host cells were used for the screens. The host cell base strain was engineered to express one or more copies of CYP1798, CYP5491, AtCPR, CPR4497, SgCDS, EPH3, and AtEPH2, as well as to upregulate expression of ERGS and ERG1 and downregulate expression of ERG7. The base strain also had several copies of pPGK1_X_tSSA1 integrated into the genome. “X” corresponds to the F-Cph1 recognition site, which is 24 bp and has the sequence GATGCACGAGCGCAACGCTCACAA (SEQ ID NO: 46).

To test the protein library for enhanced mogrol production, an in vivo plate assay was combined with LC-MS analysis. Plasmids carrying individual genes were transformed and integrated into the chromosome of a S. cerevisiae chassis strain that produces mogrol. A strain lacking any additional plant protein was used as a negative control.

Single colonies resulting from transformation were grown as pre-cultures containing culturing media in a shaking incubator at 26° C. for 96 hours at 1000 rpm. After 48 hours, pre-cultures were transferred into production media and grown in a shaking incubator at 26° C. for 96 hours at 1000 rpm. After 96 hours, cultures were extracted with an organic solvent and product formation was tested by LC-MS analysis to evaluate mogrol and mogroside production. A Thermo Scientific Q Exactive Focus MS with a LX2 multiplexed columns setup was used. Thermo Scientific Accucore PFP columns (2.6 μm, 2.1 mm×100 mm) with 12.5 mM ammonium acetate pH 8.0 in water running buffer and acetonitrile ramp were used for separation in negative mode using full scan.

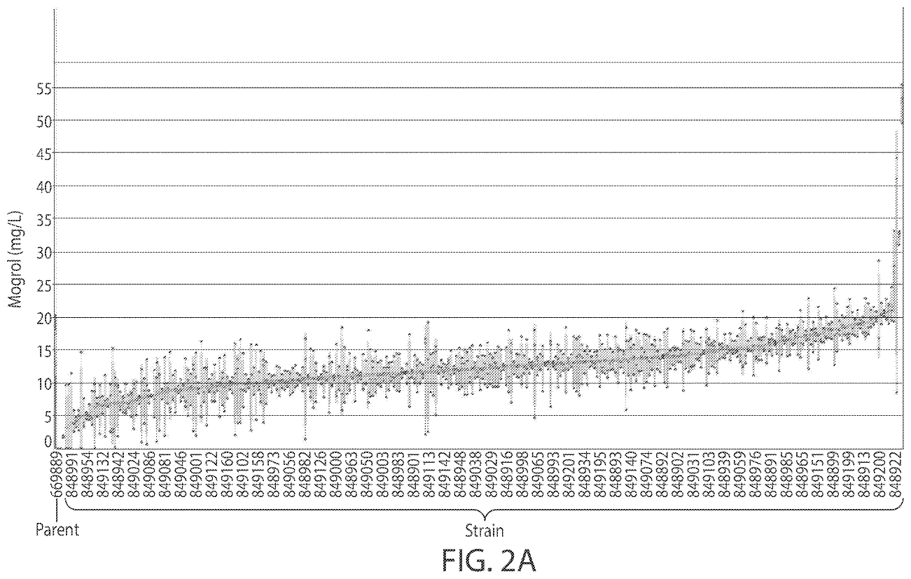

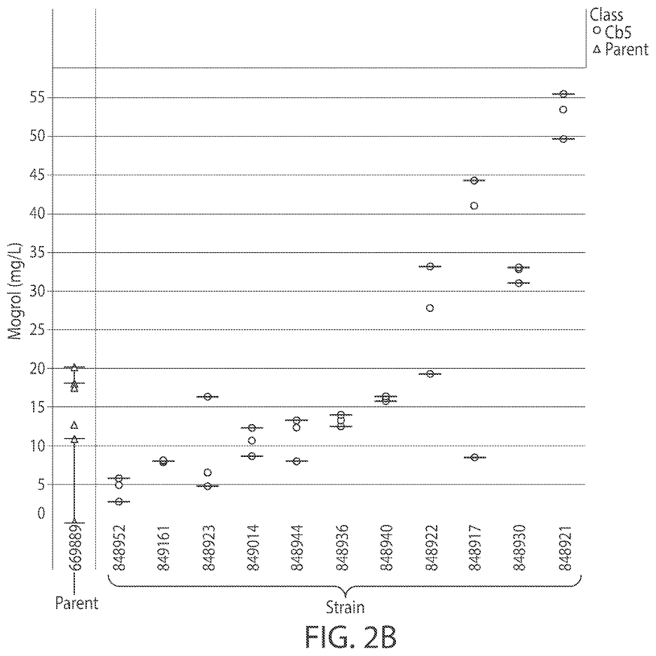

Initially, a short analytical run was performed to identify product species based on mass. Based on this screen, several proteins were identified that increased mogrol production of the parental strain (Table 3 and FIGS. 2A-2B). In particular, eleven cytochrome b5 (CB5) proteins were included in the screen (FIG. 2B). Several of these CB5 proteins were found to increase mogrol production of the parental strains, including the CB5 proteins expressed by strains 848921, 848930, 848917, 848922, and 848940.

Analysis of CB5 proteins in the screen using a motif identification software identified multiple sequence motifs that were enriched in CB5 proteins that increased mogrol production as compared to CB5 proteins that did not increase mogrol production.

The following motifs, corresponding to SEQ ID NOs: 47-49, are present in the CB5 sequences expressed in strains 848917, 848921, 848922, and 848930:

a)
the amino acid sequence
(SEQ ID NO: 47)
YTGLSP;
b) the amino acid sequence
(SEQ ID NO: 48)
KPLLMAIKGQIYDVS;
and
c) the amino acid sequence
(SEQ ID NO: 49)
LQDWEYKFM

The following motifs, corresponding to SEQ ID NOs: 50-53, are also present in the CB5 sequences expressed in strains 848917, 848921, 848922, and 848930:
a) the amino acid sequence
   X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX22X₂₃FX₂₄X₂₅X₂₆X₂₇X₂₈X₂₉X₃₀X₃₁ (SEQ ID NO: 50), in which:
      (i) X₁ is the amino acid E or Q;
      (ii) X₂ is the amino acid L or V;
      (iii) X₃ is the amino acid Y or W;
      (iv) X₄ is the amino acid W or E;
      (v) X₅ is the amino acid K or T;
      (vi) X₆ is the amino acid A or L;
      (vii) X₇ is the amino acid M or K;
      (viii) X₈ is the amino acid Q or A;
      (ix) X₉ is the amino acid A or V;
      (x) X₁₀ is the amino acid W or A;
      (xi) X₁₁ is the amino acid T or A;
      (xii) X₁₂ is the amino acid A or T;
      (xiii) X₁₃ is the amino acid I or V;
      (xiv) X₁₄ is the amino acid S or L;
      (xv) X₁₅ is the amino acid M or G;
      (xvi) X₁₆ is the amino acid I or L;
      (xvii) X₁₇ is the amino acid F or A;
      (xviii) X₁₈ is the amino acid F or Y;
      (xix) X₁₉ is the amino acid Q or Y;
      (xx) X₂₀ is the amino acid M or V;
      (xxi) X₂₁ is the amino acid V or I;
      (xxii) X₂₂ is the amino acid S or G;
      (xxiii) X₂₃ is the amino acid M or F;
      (xxiv) X₂₄ is the amino acid V or G;
      (xxv) X₂₅ is the amino acid S or T;
      (xxvi) X₂₆ is the amino acid P or S;
      (xxvii) X_{2 7} is the amino acid E or D;
      (xxviii) X₂₈ is the amino acid E or Y;
      (xxix) X₂₉ is the amino acid F or G;
      (xxx) X₃₀ is the amino acid N or S; and/or
      (xxxi) X₃₁ is the amino acid K or H;
b) the amino acid sequence
   X₁VQX₂GX₃X₄X₅EX₆X₇LX₈X₉YDGSDX₁₀X₁₁KPLLMAIKGQIYDVSX₁₂X₁₃RMF (SEQ ID NO: 51), in which:
      (i) X₁ is the amino acid P or A;
      (ii) X₂ is the amino acid V or I;
      (iii) X₃ is the amino acid E or Q;
      (iv) X₄ is the amino acid I or L;
      (v) X₅ is the amino acid S or T;
      (vi) X₆ is the amino acid E or Q;
      (vii) X₇ is the amino acid E or Q;
      (viii) X₈ is the amino acid K or R;
      (ix) X₉ is the amino acid Q or A;
      (x) X₁₀ is the amino acid S or P;
      (xi) X₁₁ is the amino acid K or N;
      (xii) X₁₂ is the amino acid Q or S; and/or
      (xiii) X₁₃ is the amino acid S or G;
c) the amino acid sequence
   LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), in which:
      (i) X₁ is the amino acid K or L;
      (ii) X₂ is the amino acid M or L;
      (iii) X₃ is the amino acid E or K;
      (iv) X₄ is the amino acid E or P;
      (v) X₅ is the amino acid K or E;
      (vi) X₆ is the amino acid L or I;
      (vii) X₇ is the amino acid D or N;
      (viii) X₈ is the amino acid S or E;
      (ix) X₉ is the amino acid G or S;
      (x) X₁₀ is the amino acid P or E;
      (xi) X₁₁ is the amino acid F or E;
      (xii) X₁₂ is the amino acid E or V;
      (xiii) X₁₃ is the amino acid A or I;
      (xiv) X₁₄ is the amino acid S or E;
      (xv) X₁₅ is the amino acid T or E; and/or
      (xvi) X₁₆ is the amino acid V or L; and
d) the amino acid sequence X₁X₂X₃EX₄GX₅X₆X₇X₈X₉X₁₀D (SEQ ID NO: 53), in which:
   (i) X₁ is the amino acid K or E;
   (ii) X₂ is the amino acid P or H;
   (iii) X₃ is the amino acid A or S;
   (iv) X₄ is the amino acid D or N;
   (v) X₅ is the amino acid P or H;
   (vi) X₆ is the amino acid S or R;
   (vii) X₇ is the amino acid E or N;
   (viii) X₈ is the amino acid S or F;
   (ix) X₉ is the amino acid Q or E; and/or
   (x) X₁₀ is the amino acid A or I.

The following motifs, corresponding to SEQ ID NOs: 58, 60, 62, and 64, are present in the CB5 sequences expressed in strains 848917 and 848921:

a)
(SEQ ID NO: 58)
QVWETLKEAIVAYTGLSPATFFTVLALGLA
VYYVISGFFGTSDYGSH;
b)
(SEQ ID NO: 60)
PVQVGEISEEELKQYDGSDSKKPLLMAIKG
QIYDVSQSRMF;
c)
(SEQ ID NO: 62)
LAKMSFEEKDLTGDISGLGPFELEALQDWE
YKFMSKYVKVGTV;
and
d)
(SEQ ID NO: 64)
KPAEDGPSESQAD

The following motifs, corresponding to SEQ ID NOs: 59, 61, 63, and 65, are present in the CB5 sequences expressed in strains 848922 and 848930:

a)
(SEQ ID NO: 59)
ELYWKAMEQIAWYTGLSPTAFFTILAS
MIFVFQMVSSMFVSPEEFNK;
b)
(SEQ ID NO: 61)
AVQIGQLTEQQLRAYDGSDPNKPLLMA
IKGQIYDVSSGRMF;
c)
(SEQ ID NO: 63)
LALLSFKPEDITGNIEGLSEEELVILQ
DWEYKFMEKYVKVGEL;
and
d)
(SEQ ID NO: 65)
EHSENGHRNFEID

The following motifs, corresponding to SEQ ID NOs: 54-57, are present in the CB5 sequence expressed in strain 848940:

a) the amino acid sequence
(SEQ ID NO: 54)
ILRVSFRKYRKAIEQ;
b) the amino acid sequence
(SEQ ID NO: 55)
RAFRPSIRFKKSHSTVPT;
c) the amino acid sequence
(SEQ ID NO: 56)
KNTLYVGG;
and/or
d) the amino acid sequence
(SEQ ID NO: 57)
DQATQKHRSFGFVTFLEKED

TABLE 3
Mogrol production by strains comprising CB5s

| | SEQ ID of | | |
|---|---|---|---|
| | CB5 expressed | Mogrol | |
| Strain | in each strain | (mg/L) | STDEV |
| Parent | N/A | 13.31 | 6.80 |
| 848921 | 1 | 52.76 | 2.91 |
| 848930 | 3 | 32.30 | 1.12 |
| 848917 | 2 | 31.29 | 19.73 |
| 848922 | 3 | 26.77 | 6.98 |
| 848940 | 4 | 16.16 | 0.29 |
| 848936 | 6 | 13.30 | 0.73 |
| 848944 | 7 | 11.28 | 2.79 |
| 849014 | 8 | 10.62 | 1.82 |
| 848923 | 5 | 9.27 | 6.22 |
| 849161 | 9 | 8.10 | 0.12 |
| 848952 | 10 | 4.58 | 1.55 |

As shown in Table 3 and in FIG. 2B, multiple CB5 proteins, which may interact with cytochrome P450 enzymes as well as cytochrome P450 reductase partners, were identified in this screen that resulted in enhanced mogrol production.

### Example 2. Increased Mogrol Production by CB5 Proteins in Y. lipolytica

This Example describes testing representative S. grosvenorii cytochrome b5 proteins identified in Example 1 in Y. lipolytica to confirm that the proteins enhance mogrol production in multiple cell types.

Three CB5 proteins identified in Example 1 (corresponding to SEQ ID NO: 1, SEQ ID NO: 2, and SEQ ID NO: 3), and a truncated version of SEQ ID NO: 1 and SEQ ID NO: 3 (corresponding to SEQ ID NO: 318), were expressed in Y. lipolytica host cells to determine whether the proteins enhanced mogrol or mogroside production in Y. lipolytica.

Two different host cell base strains were engineered to express one or more copies of CYP1798, CYP5491-T351M, AtCPR, SgCDS, and EPH3, as well as to upregulate expression of ERG1 and downregulate expression of ERG7.

To test the strains expressing the CB5 proteins for enhanced mogrol production, an in vivo plate assay was combined with LC-MS analysis. Plasmids carrying individual genes were transformed and integrated into the chromosome of Y. lipolytica parent strains that produce mogrol. Parent strains lacking any S. grosvenorii cytochrome b5 protein were used as negative controls, corresponding to strains 974137 and 1419596. Single colonies resulting from transformation were grown as pre-cultures containing culturing media in a shaking incubator at 30° C. for 96 hours at 1000 rpm. After 48 hours, pre-cultures were transferred into production media and grown in a shaking incubator at 30° C. for 96 hours at 1000 rpm. After 96 hours, cultures were extracted with an organic solvent and product formation was tested by LC-MS analysis to evaluate mogrol and mogroside production. A Thermo Scientific Q Exactive Focus MS with a LX2 multiplexed columns setup was used. Thermo Scientific Accucore PFP columns (2.6 μm, 2.1 mm×₁₀₀ mm) with 12.5 mM ammonium acetate pH 8.0 in water running buffer and acetonitrile ramp were used for separation in negative mode using full scan. Initially, a short analytical run was performed to identify product species based on mass.

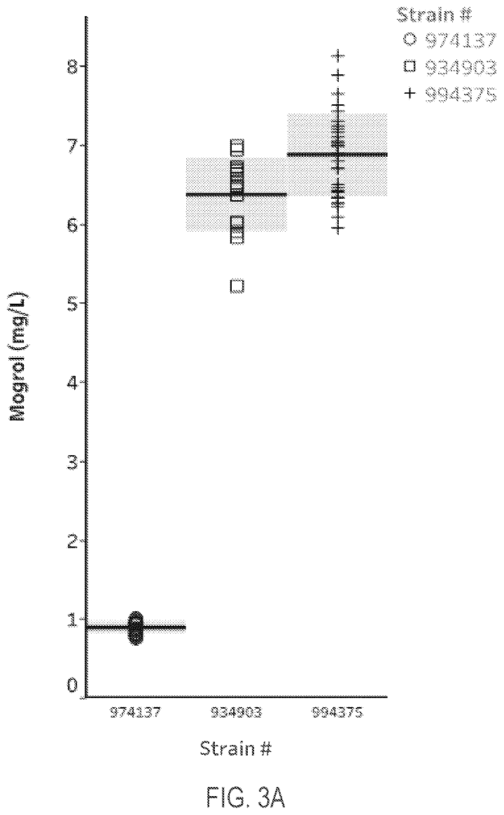

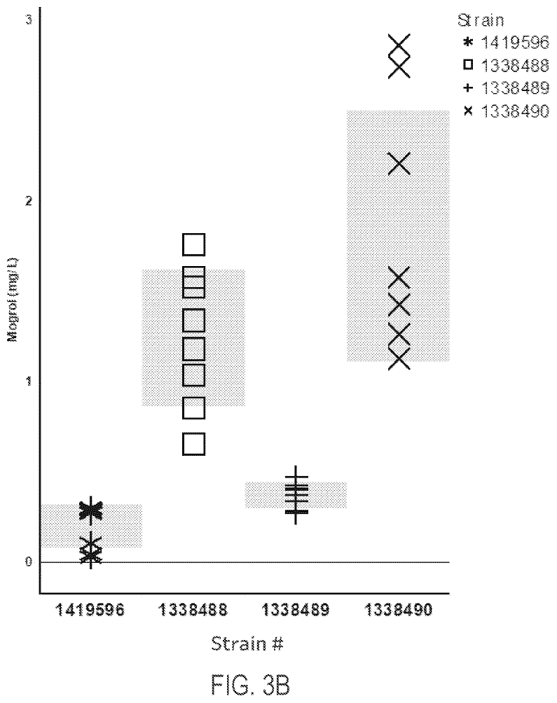

The CB5 protein with a sequence corresponding to SEQ ID NO: 1 as well as the truncated form, CB5-trunc, with a sequence corresponding to SEQ ID NO: 318, expressed in strains 994375 and 934903 respectively, were observed to increase mogrol production relative to the parental strain 974137 in a first strain background (Table 4 and FIG. 3A).

In a second strain background, the CB5 proteins with a sequence corresponding to SEQ ID NO: 1, SEQ ID NO: 2, or SEQ ID NO: 3 expressed in strains 1338488, 1338490, and 1338489, respectively, were observed to increase mogrol production relative to the parental strain 1419596 (Table 4 and FIG. 3B).

Motifs corresponding to SEQ ID NOs: 47-49, 50-52, 58, 60, and 62, discussed above, are present in the CB5 sequences expressed in strains 994375, 934903, and 1338490.

TABLE 4
Mogrol production by Y. lipolytica strains comprising CB5 proteins

| | | | |
|---|---|---|---|
| | SEQ ID of CB5 | | |
| | expressed in | Mogrol | |
| Strain | each strain | (mg/L) | STDEV |

Parent 1

| | | | |
|---|---|---|---|
| Parent 1 (974137) | N/A | 0.90 | 0.03 |
| 934903 | 318 | 6.38 | 0.47 |
| 994375 | 1 | 6.88 | 0.52 |

Parent 2

| | | | |
|---|---|---|---|
| Parent 2 (1419596) | N/A | 0.20 | 0.12 |
| 1338488 | 1 | 1.24 | 0.38 |
| 1338489 | 3 | 0.37 | 0.07 |
| 1338490 | 2 | 1.81 | 0.70 |

As shown in Table 4 and in FIGS. 3A-3B, this Example shows that representative CB5 proteins identified in Example 1, and a non-naturally occurring truncated form of a CB5 protein sharing similarity to CB5 proteins identified in Example 1, were able to enhance mogrol production in Y. lipolytica host cells, confirming that this effect is not limited to S. cerevisiae cells. These data indicate that the identified CB5 proteins are able to enhance mogrol production through interactions with the heterologous pathways expressed in the host cells and that this effect is not host-dependent.

TABLE 5
Non-limiting examples of CB5 sequences

| | | SEQ | | SEQ |
|---|---|---|---|---|
| | | ID | Amino Acid | ID |
| Strain | Nucleotide Sequence | NO | Sequence | NO |
| 848921 | atggccttgcaggtgtgggaaaccttaaaagaggctatt | 11 | MALQVWETLKEAIVAYTG | 1 |
| | gtagcttatacggggctttcccctgcaacatttttcact | | LSPATFFTVLALGLAVYY | |
| | gtcctagctttgggtctggcggtttactacgttatctca | | VISGFFGTSDYGSHSRDF | |
| | ggtttctttggaacctctgattatggttctcacagtcgt | | GEQMQPLPPPVQVGEISE | |
| | gacttcggtgaacaaatgcaaccettaccaccaccagtc | | EELKQYDGSDSKKPLLMA | |
| | caagttggcgaaatatccgaagaggaattgaagcaatac | | IKGQIYDVSQSRMFYGPG | |
| | gacggtagcgattcaaagaagccattgctaatggctatc | | GPYALFAGKDASRALAKM | |
| | aaaggccaaatttacgatgtctcgcaatctaggatgttc | | SFEEKDLIGDISGLGPFE | |
| | tatggtcccggtggtccatacgccctctttgctggtaag | | LEALQDWEYKFMSKYVKV | |
| | gacgcttctcgagctttggcaaaaatgtccttcgaagaa | | GTVEKPVSETDAAPAGES | |
| | aaggacttgactggtgatatttctggtttgggccctttc | | AESTNAEVSKPAEDGPSE | |
| | gaattagaagccttacaggattgggaatacaagtttatg | | SQADKAEETVAAKVE | |
| | tccaaatacgttaaggtcggtactgttgaaaagccagtt | | | |
| | tcagagacagatgctgcccctgctggtgaatctgcggag | | | |
| | tctactaacgctgaagtctcgaaaccagcagaagacggt | | | |
| | ccttctgaatcccaagctgataaggctgaagagactgta | | | |
| | gccgctaaggttgaatag | | | |
| 848917 | atggccttgcaggtatgggagactctaaaagaagcaatt | 12 | MALQVWETLKEAIVAYTG | 2 |
| | gtcgcttataccggtcttagtccagctacgttctttaca | | LSPATFFTVLALGLAVYY | |
| | gtgttggctctgggattagccgtttactacgtcatatct | | VISGFFGTSDYGSHSRDF | |
| | ggtttctttggtacctcggactatggctcacattctcgt | | GEQMQPLPPPVQVGEISE | |
| | gatttcggggaacaaatgcaacctttgccaccgccagtt | | EELKQYDGSDSKKPLLMA | |
| | caagttggtgaaatctccgaagaggaattaaagcaatac | | IKGQIYDVSQSRMFYGPG | |
| | gatggttctgacagcaagaagccattgttgatggctatt | | GPYALFAGKDASRALAKM | |
| | aaaggtcaaatctacgacgtttcccaatctaggatgttt | | SFEEKDLTGDISGLGPFE | |
| | tacggtccaggtggtccctatgcgcttttcgctggcaag | | LEALQDWEYKFMSKYVKV | |
| | gatgcctcgcgcgctttggctaagatgagttttgaagaa | | GTVEKPVSETDAAPAGES | |
| | aaagacctgactggggatatttcaggattaggccctttc | | AESTNAEVSKPAEDGPSE | |
| | gaattggaagctctccaggattgggaatataagttcatg | | SQADKAEETVAAKQKKEK | |
| | tctaagtacgtcaaagtgggtactgttgaaaagccagtt | | KKGRRRVEGVLMEIRGEA | |
| | tccgagacagacgcagccccagctggtgaatctgccgaa | | VGGRKGEGMSREIGRGGG | |
| | agtaccaacgctgaagtctccaaacctgctgaagatggt | | GADDGNLKGGGIDHGERE | |
| | ccaagcgaatcccaagctgacaaggcggaggaaactgta | | RERERESGMS | |
| | gccgctaagcaaaaaaaggaaaaaaagaagggtagaaga | | | |
| | agagttgaaggtgtcctaatggaaatcagaggtgaagcg | | | |
| | gttggtgggagaaagggtgagggaatgtcaagagaaatc | | | |
| | ggtcggggtggtggtggtgctgatgatggtaatttgaaa | | | |
| | ggtggcggtattgaccacggtgaacgtgagcgagagaga | | | |
| | gaaagagagtctggtatgtcttag | | | |
| 848922 | atggaattgtactggaaagcaatggagcagattgcctgg | 13 | MELYWKAMEQIAWYTGLS | 3 |
| | tataccggcttatctcctacagctttctttactatactg | | PTAFFTILASMIFVFQMV | |
| | gcttcgatgatcttcgtgtttcaaatggtcagttccatg | | SSMFVSPEEFNKPPTVPV | |
| | ttcgtttctccggaagaatttaataagcccccaactgtc | | SSSNPANSNLFVNDSVAD | |
| | ccagtttcctcatctaacccagctaactctaatctattc | | ASQAVQIGQLTEQQLRAY | |
| | gtaaacgacagcgttgcggatgccagccaagctgttcaa | | DGSDPNKPLLMAIKGQIY | |
| | attggtcaattgacggaacaacagttgagggcttacgat | | DVSSGRMFYGPGSPYAMF | |
| | ggatccgacccaaacaagccattacttatggctatcaaa | | AGKDASRALALLSFKPED | |
| | ggtcaaatttatgatgtctctagtggtagaatgttctac | | ITGNIEGLSEEELVILQD | |
| | ggtcctggtagtccatacgcaatgttcgccggcaaggac | | WEYKFMEKYVKVGELVLE | |
| | gcttcaagagctttggcattgctctcttttaagccagaa | | GGGEGMNEHSENGHRNFE | |
| | gacatcaccggtaacattgaaggtttgtcagaagaggaa | | IDQEEERIEAR | |
| | ttagttatcttacaagattgggagtacaagttcatggaa | | | |
| | aagtatgttaaagtgggggaattggttctcgaaggtggt | | | |
| | ggtgaaggtatgaatgaacactctgagaacggccatcgt | | | |
| | aattttgaaatcgatcaagaagaagaaagaattgaggct | | | |
| | agatag | | | |
| 848930 | atggaactttattggaaggctatggagcaaatagcctgg | 14 | MELYWKAMEQIAWYTGLS | 3 |
| | tacacagggttatcgcctacggcattctttactattcta | | PTAFFTILASMIFVFQMV | |
| | gctagtatgattttcgtctttcagatggtgtcatctatg | | SSMFVSPEEFNKPPTVPV | |
| | ttcgttagcccagaagaatttaataaaccaccaaccgta | | SSSNPANSNLFVNDSVAD | |
| | cccgtttcctcttccaaccctgctaactctaatttgttc | | ASQAVQIGQLTEQQLRAY | |
| | gtcaacgatagcgttgctgacgcctctcaagcggtccaa | | DGSDPNKPLLMAIKGQIY | |
| | atcggtcaattgaccgaacaacaattgcgtgcttacgat | | DVSSGRMFYGPGSPYAMF | |
| | ggttccgacccaaacaagccattactgatggccatcaag | | AGKDASRALALLSFKPED | |
| | ggtcagatttatgatgtttcctctggaaggatgttctac | | ITGNIEGLSEEELVILQD | |
| | ggcccgggttcaccatacgctatgttcgccggtaaagac | | WEYKFMEKYVKVGELVLE | |
| | gcgtcgagagctctcgctttgttgtcctttaagccagaa | | GGGEGMNEHSENGHRNFE | |
| | gatatcactggtaacattgagggtttgagtgaagaagaa | | IDQEEERIEAR | |
| | ttggttatcttacaagactgggaatacaagttcatggag | | | |
| | aagtatgtgaaagtcggtgaacttgtcttggaaggtgga | | | |
| | ggtgaaggtatgaatgaacattcagagaatggccacaga | | | |
| | aactttgaaattgatcaggaagaagaaagaatcgaagct | | | |
| | agatag | | | |
| 848940 | atgaccgccactatcacagcacagcaccgtaacggctgc | 15 | MTATITAQHRNGCGPSPP | 4 |
| | ggaccttccccacccaaaaagttggacattctgagagta | | KKLDILRVSFRKYRKAIE | |
| | agtttccgaaagtatagaaaggctatagaacaaccaacg | | QPTEAISRLIAKRDRCRA | |
| | gaggctatttcgagactaattgctaaaagagatcgctgt | | FRPSIRFKKSHSTVPTTI | |
| | agagcgtttaggccatctatcagattcaagaaatcacat | | VYDLTSYLDEHPGGDDVI | |
| | tccaccgtgccaactactatcgtttacgatttgacctct | | LAATGRDATDDFEDAGHS | |
| | tacttagacgaacaccctggtggtgatgacgttatcttg | | KDARELMQKFYIGLLDTS | |
| | gccgccactggtcgtgatgctacagacgattttgaagat | | SSASPELETKQPRGYAAR | |
| | gctggtcattctaaggatgctagagaacttatgcaaaag | | VQGLTKQYWAAPVAILGI | |
| | ttctacattggtttattggacaccagctcttccgcttcc | | SLAVQKNTLYVGGLAEEV | |
| | ccagagttggaaacgaaacaaccgagaggttatgctgca | | NESILHAAFIPFGDIKDV | |
| | agagtccaagggttgactaagcaatactgggcagctcca | | KTPLDQATQKHRSFGFVT | |
| | gttgccatactcggtatttcattagccgtccaaaagaat | | FLEKEDASAAMDNMDGAE | |
| | actttgtacgtcggcggtctggctgaagaagttaacgaa | | LYGRVLTVNYALPERIKG | |
| | agtatcttgcacgctgctttcattccttttggtgacatt | | GEQGWAAQPIWADADTWF | |
| | aaagacgttaagaccccgttagaccaagctacccagaag | | ERQQQEEEMQRIQAENRA | |
| | catagatcattcgggtttgttactttcctagaaaaggaa | | AMQAAEELHRKKLAQERE | |
| | gatgcctctgcggctatggataatatggacggtgcggaa | | GEKEDEIDTKDDPMAKAE | |
| | ttgtatggtcgtgtcttgactgtaaactacgccttgcca | | AEVLRQNS | |
| | gagagaatcaagggtggagaacaaggttgggctgcgcaa | | | |
| | ccaatctgggctgatgctgatacctggttcgagagacaa | | | |
| | caacaagaagaagaaatgcagagaatacaagccgaaaac | | | |
| | cgcgccgctatgcaagctgctgaagagctacacagaaaa | | | |
| | aagcttgctcaggaaagagaaggtgaaaaagaagatgaa | | | |
| | attgataccaaagacgacccgatggccaaggcagaagct | | | |
| | gaggtcctacgacaaaatagttag | | | |
| 848923 | atggttggaaaggccatgaggagaaatagagctcataaa | 16 | MVGKAMRRNRAHKDRVEN | 5 |
| | gatcgtgtcttcaactttttgctgccagaccctaaggta | | FLLPDPKVFVFDEVAKHN | |
| | ttcgtttttgatgaggttgcaaagcacaaccacttaaga | | HLRDCWIIISGKVYDVTP | |
| | gactgctggatcataatttcaggcaaagtctatgatgtt | | FLEEHPGGDEVLLLAAEK | |
| | acaccattcttggaagaacatccgggtggtgacgaagtg | | DATDDFENVGHSLAAIEQ | |
| | cttctattagctgctgaaaaggatgcgactgacgatttt | | MEKYYVGKVDMSTVPKPI | |
| | gaaaatgtcggtcacagcttggctgccatcgaacaaatg | | DYRPPASHPATTPSAQSS | |
| | gagaagtactacgttggtaaagtcgacatgtccacggtc | | GSLIRLLQFLIPLLILGV | |
| | ccaaagccaattgactatcggccccctgcttctcatcca | | AFYLQFYGKKQQSN | |
| | gctaccaccccatctgcccagagttctggttcgttgatt | | | |
| | cgattgcttcaattcttgatcccattattgattttaggt | | | |
| | gttgctttctacttacaattttacgggaagaaacaacaa | | | |
| | tccaactag | | | |
| 848936 | atgaaggattttcaggacgagatcgcagccagtggtgct | 17 | MKDFQDEIAASGAVPAAD | 6 |
| | gtccctgctgctgaccacaaactactgcgtacagaaccc | | HKLLRTEPAECMAAWRQR | |
| | gctgaatgcatggccgcatggagacaacgcagagctttg | | RALVPHHANAAAELSASS | |
| | gtgccacatcacgccaacgctgctgccgaattgtctgct | | VVFLFVIVVFVGVVVEIF | |
| | agctcagttgttttcttgttcgttatagtagtctttgtt | | FVAHSSISDILTGCDVGD | |
| | ggcgtcgttgtcgaaattttcttcgttgcgcattcttcc | | DSDPVVDSSLSFSSSLFS | |
| | atttctgatatcttaaccggttgtgacgttggtgatgat | | SCSFSSSSAASAAAAALL | |
| | tccgacccagtagtggattcgtctttgtccttttctagc | | LHRLLLVLLRLPSRTLFS | |
| | agtcttttctcttcctgttctttttcctcatcatcggcc | | PPIRRCLSVGLGCRVLVL | |
| | gctagtgctgcggctgctgcattattgctccacaggttg | | RLERGSIRTSHRAGTSLN | |
| | ttacttgttttgttgagactaccatctcggactttgttc | | RRSCSVWNSGEMFGDGKR | |
| | tcgccacctattagaagatgtttgtccgtcggacttggt | | ISTILGTVETQAEPARFS | |
| | tgtagagtcttagttttaagattggaaagaggttctatt | | RSLSLKDFQVLLSLPKRP | |
| | cgaacttcacatagagctggtacgtctttaaatagaaga | | LPALGSSSRIMSSRVRLR | |
| | tcatgttccgtatggaactcaggggaaatgttcggtgac | | ISAARPVAGGPSLFRRQP | |
| | ggtaagcgtatcagcaccattctagggactgttgagaca | | PSVLPIEAGHRKKRKCQM | |
| | caagcagaaccagcaagattttctcgctctttgtccttg | | ELDAGMASARTWELGRRK | |
| | aaggatttccaagtcctactgagcttgcctaaaagacca | | KKKKIFRSKFENLESKMQ | |
| | ttgccagctttgggtagctcttcccgtatcatgtcttct | | DEEKRGSGWCENAIASAS | |
| | cgcgtcagactgagaataagtgcggccagaccagttgct | | PTHTQHSIDCRTSLPLFH | |
| | ggtggtccgagtctattcaggcgacaaccaccgtctgta | | AATMGGRSVYTLAEVASH | |
| | ctgccaatcgaagctggacacagaaagaaaagaaagtgc | | DNRNDCWLIIENKVYDVT | |
| | caaatggaattggacgctggtatggcctccgctagaact | | KFLEDHPGGDEVLLSAIG | |
| | tgggagttaggtagacgtaagaagaaaaagaaaattttt | | KDATNDFDDVGHSSAARA | |
| | agaagcaagttcgaaaacttggaatctaagatgcaggat | | MMDEFYVGDIDSSTIPAK | |
| | gaagaaaaaagaggtagtggttggtgtgagaatgctatc | | RKYTPPKQPLYNQDKTPE | |
| | gcctccgcttctccaacccacacccaacactcaattgac | | FIIKVLQFLVPLVCSHRP | |
| | tgccggacgtccctaccactctttcatgccgcgactatg | | RKNLTTHCGALVAQNTGL | |
| | ggcggtagatccgtttacactttggctgaagttgccagt | | EPCTGGEGAHFEQHEHQP | |
| | catgataatagaaacgattgttggttaattatagaaaac | | LARASGNQLVCRASSCLS | |
| | aaggtttatgatgttactaagttcttagaagaccatcca | | LIDPDEPVKVIKSSNIQL | |
| | ggtggcgacgaagtcttgttgtccgctatcggtaaggat | | GSYPSLEVKSASNSAIKH | |
| | gctaccaatgactttgacgatgtgggtcacagttccgct | | HSTSQKPEKEIDWNSQFF | |
| | gctagagctatgatggatgaattttacgttggtgacatc | | NWQKADYCLNNNLPELLY | |
| | gattcttctactattcccgccaagcgtaaatacacccct | | SKREKLEAICSEQAFDAI | |
| | cctaagcaaccattgtataaccaagacaaaaccccagaa | | ASMQRLTTSAHRQEGVNS | |
| | tttattataaaggtacttcaattcttggtccctctagtt | | HIAATLQHTFQGSWRRYS | |
| | tgttctcatagaccaagaaaaaatcttacaactcactgc | | QDILQLANQDYPLRTLQS | |
| | ggagcactcgtcgcgcaaaacaccggtttggaaccatgt | | FQDNQIHNIAAQHFLE | |
| | actggcggggaaggtgctcatttcgaacagcacgaacac | | | |
| | caacccttggccagagcttctggtaatcaattggtttgt | | | |
| | cgggcttcctcctgtctctctttgatcgatccagatgag | | | |
| | ccagttaaggttatcaagtcttctaacattcaactaggt | | | |
| | tcatacccctccttggaggtgaaaagtgcttcgaacagt | | | |
| | gctatcaagcaccatagtacatctcagaagcctgaaaaa | | | |
| | gaaatagattggaatagccaattcttcaactggcaaaag | | | |
| | gctgattattgtttaaacaacaatctgccagaattgtta | | | |
| | tacagcaaaagagaaaagttggaagctatttgctctgaa | | | |
| | caagcttttgacgcaattgcctccatgcaaagactaact | | | |
| | acaagcgcccaccgtcaagaaggtgttaactctcacata | | | |
| | gcggccactctacaacacacgtttcaaggttcgtggaga | | | |
| | agatactcacaagatatcttgcaattagccaaccaagac | | | |
| | tatccattgagaaccttgcaaagtttccaagacaaccaa | | | |
| | attcataatattgctgctcaacatttcctcgaatag | | | |
| 848944 | atggcctctgatccaaaggtgcaagtcttcgaagaggtt | 18 | MASDPKVQVFEEVAKHNK | 7 |
| | gctaaacacaacaagcctaaaattgtctacgacgtaacc | | PKIVYDVTPFMEDHPGGD | |
| | ccatttatggaagatcatccaggaggtgacgaagtttta | | EVLLSATGKDATNDFEDV | |
| | ctttccgcaactggtaaggatgctacaaatgacttcgaa | | GHSDSAREMMDKYYIGEI | |
| | gatgttggccactcagattcggcgagagaaatgatggac | | DPSTVPLKKIYIPTQQTQ | |
| | aagtattacatcggtgaaatagaccccagcactgtcccg | | YTPDKTPEFVIKILQILV | |
| | ctgaagaaaatttacatcccaacccagcaaactcaatat | | PILILGLAFAVRHYTKNE | |
| | acgcctgacaagactccagagtttgttataaagattttg | | | |
| | caaatcttggtcccaattttgatcttaggtttggctttc | | | |
| | gctgttcgtcattacaccaagaacgaatag | | | |
| 849014 | atgccttcaatctctacgctctactcgatacaggaggta | 19 | MPSISTLYSIQEVSQHST | 8 |
| | tctcaacatagtacaaatgatgactgctggattgtgatt | | NDDCWIVIDGKVYDLTSY | |
| | gatggcaaggtttatgacttgacttcctacctagatgaa | | LDEHPGGDDVILAATGRD | |
| | cacccgggtggagatgacgttatcttagctgccaccggt | | ATDDFEDAGHSKDARELM | |
| | cgtgatgcaaccgacgactttgaagatgctggtcactcc | | QKFYIGLLDTSSSASPEL | |
| | aaagacgctagagaattgatgcaaaagttctatatcggt | | ETKQPRGYAARVQGLTKQ | |
| | cttttagacactagctcttcagcgtcccccgaattggag | | YWAAPVAILEKTTTKKNL | |
| | actaagcaaccaagagggtacgccgctagggtccaaggt | | KELQKWRINASNGAGSSE | |
| | ctgaccaaacaatactgggccgcaccagtcgctattttg | | KHFVRRWFGGGGERVYPT | |
| | gaaaagactaccactaagaaaaacttgaaggaattgcaa | | CSLHTLRRHQRRQDSIGS | |
| | aagtggagaattaacgcctctaatggtgctggatcttcc | | SHPEAPFLRLRHFLGKED | |
| | gaaaagcatttcgttcggcgctggtttggtggtggtggt | | ASAAMDNMDGAELYGRVL | |
| | gaaagagtctatccaacatgttcactacataccttgaga | | TVNYALPERIKGGEQGWA | |
| | agacaccagcgtagacaagacagtataggtagctctcac | | AQPIWADADTWFERQQQE | |
| | ccagaggctcctttcctgagattacgtcacttcctaggc | | EEMQRIQAENRAAMQAAE | |
| | aaagaagatgcctccgcagcgatggataacatggatggt | | ELHRKKLAQEREGEKEDE | |
| | gctgaactttacggtagagttttaactgttaactacgct | | IDTKDDPMAKAEAEVLRQ | |
| | ttgccagaaagaatcaagggtggtgaacaaggatgggct | | NS | |
| | gcccaaccaatttgggctgacgctgatacatggtttgaa | | | |
| | agacaacaacaagaagaggaaatgcaaaggatccaagcc | | | |
| | gaaaacagagctgctatgcaggctgccgaagaattgcat | | | |
| | agaaaaaagttggctcaggaaagagagggtgagaaggaa | | | |
| | gatgaaattgacactaaagatgatccaatggctaaagca | | | |
| | gaagctgaagttttacgacaaaattcttag | | | |
| 849161 | atgaccgtcagagagatagttgacatttcaaacctagct | 20 | MTVREIVDISNLAESCTS | 9 |
| | gaatcctgcacttctcttttatgtcgtaggcaaaataac | | LLCRRQNNRFVHIAARFV | |
| | agattcgtgcatatcgccgcaagatttgttgttgatgaa | | VDEDVEGVWLEELLEALT | |
| | gatgtagaaggagtctggctggaagaattgttggaagct | | LVCEEGIRLSLSDASCQG | |
| | ttgacgttggtttgtgaagaaggtctaagactcagctta | | RSVLIYLREDAHERERLS | |
| | tcggacgcttcttgtcagggtagatccgtcttgatctac | | FSSVKRWRERSCEPRPPL | |
| | ttgcgagaggatgcccacgaacgcgaaagactctctttc | | SGFWRLWWQLLRFSSASP | |
| | tctagtgttaaaagatggcgtgagagaagctgtgaaccc | | SNPDIRSPPPPPSSPLNR | |
| | aggccacctttatctgggttttggagattatggtggcaa | | SVFDVTKGKSHYGVGGGY | |
| | ttgttgagattctccagtgcttctccatccaatccagac | | NHFAGRDASRAFVSGNFT | |
| | attagatcaccgcctccaccaccctcctctccactgaac | | GEGLTDSLRGLSNAEIKS | |
| | agaagcgttttcgatgtcacaaagggtaagtctcactat | | VVEWRNFYNKTYTFVGKL | |
| | ggcgtaggtggtggttacaaccattttgcgggcagagat | | VGLHYDDQGNPTKHLKGA | |
| | gctagtagagccttcgtttccggtaattttactggtgaa | | EAKAARGAQLLKKQKEEE | |
| | ggtctaaccgactctttgcgtggtttgagtaacgctgaa | | DKLPSCNSRWSQGEGGEV | |
| | attaaaagtgttgtggagtggagaaacttctacaataag | | WCDDGFPRLVQRPLEIAL | |
| | acttataccttcgtcggaaagttagttggtttgcactac | | SGKMSKRCACFREDQLAE | |
| | gacgaccaaggtaacccaacaaagcatttgaaaggtgct | | PGLEVYEAALFPKRTNDD | |
| | gaagctaaggctgccaggggtgcacaactattgaagaaa | | GAAKILPPSDPCCFSCFP | |
| | caaaaggaagaagaagataaattgccctcttgcaactcc | | SGWCAGSIRNWISKSISS | |
| | agatggtcacaaggagaaggtggtgaggtctggtgtgat | | DFNTTTLPQQFLYVRSFP | |
| | gacggttttcctcggcttgttcagcgcccattagaaatc | | EGDKNCYIIKLAQGKGYK | |
| | gccttgtccggcaagatgtctaagagatgtgcttgtttc | | YLIRASFMYGNYDGQGKA | |
| | cgtgaagaccaacttgctgaaccagggttggaagtctac | | PAFDLHMGVNKWDSVILN | |
| | gaggcagctctttttcctaagagaactaatgatgatggc | | NESSIIIKEVIHALPTSS | |
| | gctgcaaagattttgcctccatcagatccatgctgtttc | | ICICLVNTGFGSPFISAL | |
| | agctgcttcccatccggttggtgtgccggttccatacgt | | ELRLLKNATYVTDFELLA | |
| | aactggatcagcaaatctatttcttctgactttaatacc | | LHRRLDIGSTINKTVRYN | |
| | actacattgccacaacaatttctctatgtcagaagtttc | | DDDCDRIWLPFNFPNYKI | |
| | ccagaaggtgataaaaactgttacataatcaagttggcg | | VSASSTVDSGVTALMNIK | |
| | cagggtaagggatataaatacttaatccgggcttcattc | | SLYGVRKNWQGDPCMPKS | |
| | atgtacggtaactatgacggtcaaggtaaggctccagcc | | YVWHGLNCSYDSHSPNRI | |
| | ttcgacttacacatgggggttaataagtgggattcggtt | | TSLNLSSSRLVGETAAYV | |
| | attttgaacaatgaatcatctatcattattaaggaagtc | | SELTSLQYLDLSNNSLSG | |
| | atccacgctttaccaacttcttctatatgtatttgtttg | | PVPGFLSELHSLKVLDLR | |
| | gtgaataccggtttcggtagcccattcatttccgccctc | | DNTLLGSIPSELMERSTN | |
| | gaactaagattgttaaagaacgccacttacgtcaccgat | | VKEIDGSDYYSNLSLDIV | |
| | tttgaattacttgctttgcatcgtcgtttagacatcggt | | NCFMEGLEETPIFVLHLL | |
| | tccacgactaacaaaactgttagatacaatgatgacgat | | AKTRRRVTLFQSNCSNSF | |
| | tgcgacagaatctggttgcctttcaacttccctaattat | | IIAGPLSCNSSLDHEMMK | |
| | aagatagtctctgcttcttcaaccgtggatagcggogtt | | TRYYASGRLTEKSDVYSF | |
| | acggctctgatgaacattaagtctttgtacggtgttaga | | GALILEIITSRPVLKINR | |
| | aagaactggcaaggtgacccatgtatgccaaaatcctat | | ASSEKCHVGQWAMHLMKT | |
| | gtctggcacggtttgaactgttcgtatgattcgcactca | | GDIRSIVDERLRGNFDLS | |
| | ccgaacagaattacttctcttaatttgagctcgtctagg | | SAWKAVEIAMTCLSQTSI | |
| | ctggttggtgaaactgctgcttacgtcagtgaattaacc | | ERPSMKEVVMELSECLAL | |
| | tccttgcaatacttggatctaagtaacaactctttgtcg | | EKARKRKNIDSNTRSSNA | |
| | ggtcccgtgccgggttttctttccgaattacattccttg | | VSRNFSESEVTPLAR | |
| | aaggttctggatttgagagacaataccttgcttggatct | | | |
| | atcccatccgaattgatggaaagatcgacaaacgtaaaa | | | |
| | gagatcgacggttctgattactactctaatttaagcctt | | | |
| | gatattgttaattgttttatggaaggattggaagagaca | | | |
| | ccaatctttgtcttgcatcttttggcaaagactagaaga | | | |
| | agagttactttattccaatccaactgttctaattcgttc | | | |
| | attatcgcgggcccactctcttgtaactcaagcttggac | | | |
| | catgaaatgatgaagaccagatattatgcatctggcaga | | | |
| | ttaacagaaaagagtgacgtatactctttcggggctttg | | | |
| | attttggagattatcacttcaaggccagtcttgaaaatc | | | |
| | aaccgtgcttccagcgaaaaatgccacgttggtcaatgg | | | |
| | gcaatgcacctcatgaaaactggtgatatcagatcaatt | | | |
| | gttgatgagagactcagaggtaactttgacctgtcctct | | | |
| | gcttggaaggctgttgaaattgcaatgacctgtctatct | | | |
| | cagacatcaatagaaagaccctctatgaaggaagtggtc | | | |
| | atggaattatctgaatgtcttgctttggaaaaggcccgt | | | |
| | aagagaaaaaatattgattctaacactagaagctccaac | | | |
| | gctgttagtagaaacttcagtgaaagtgaagtgacgcca | | | |
| | ttggccagatag | | | |
| 848952 | atgggctctggtgagaaagtgtttacactgaaggaagtc | 21 | MGSGEKVFTLKEVAEHNN | 10 |
| | gctgaacataacaatcaaaaggactgctggctagtaatc | | QKDCWLVISGKVYDVTKF | |
| | agcggtaaggtttatgatgttaccaaattcttagacgat | | LDDHPGGDDVLLSATGKD | |
| | cacccaggtggagatgacgttttgctttcggcaactggt | | ATDDFEDVGHSDNAREMM | |
| | aaggatgcgactgacgatttcgaagacgtcggtcactcc | | DQYYVGEIDASTIPKKVA | |
| | gataacgctcgtgaaatgatggaccagtactacgtcggt | | YAPPKQPHYNQDKTSEFI | |
| | gagattgatgcttcaacgatacctaagaaagttgcctat | | IKLLQFLVPLAILGLAVA | |
| | gcccccccaaagcaaccacattacaatcaagacaaaacc | | IRFYTKQA | |
| | tccgaatttattatcaagttgttgcaattcttggttccg | | | |
| | ctcgctatcttagggttggctgttgccattagattctac | | | |
| | actaagcaagcttag | | | |
| | atggctctgcaagtctgggagactctcaaggaggccatt | 22 | MALQVWETLKEAIVAYTG | 1 |
| | gtcgcttacaccggtctgtctcccgctaccttcttcact | | LSPATFFTVLALGLAVYY | |
| | gtgctcgctcttggtttggccgtctactacgtcatttcg | | VISGFFGTSDYGSHSRDF | |
| | ggcttcttcggcacctcggactacggctctcactctcga | | GEQMQPLPPPVQVGEISE | |
| | gatttcggtgagcagatgcagcccctgcctcctcccgtc | | EELKQYDGSDSKKPLLMA | |
| | caggttggtgagatctctgaggaggagctcaagcagtac | | IKGQIYDVSQSRMFYGPG | |
| | gacggcagtgactccaagaagcctcttctcatggccatc | | GPYALFAGKDASRALAKM | |
| | aagggccagatttacgatgtcagccagagccgaatgttc | | SFEEKDLTGDISGLGPFE | |
| | tacggacccggtggcccttacgctctgtttgccggcaag | | LEALQDWEYKFMSKYVKV | |
| | gatgcttctcgagctctggccaaaatgtcatttgaagag | | GTVEKPVSETDAAPAGES | |
| | aaggacctgactggtgacatttccggactcggcccattc | | AESTNAEVSKPAEDGPSE | |
| | gagcttgaggccctccaggattgggaatacaagttcatg | | SQADKAEETVAAKVE | |
| | tccaagtacgttaaggtcggcaccgttgagaagcccgtc | | | |
| | agcgagacagacgctgctcccgccggtgagtctgctgag | | | |
| | tccaccaacgccgaggtgtccaagcccgctgaggacggt | | | |
| | ccttccgagtcccaggccgacaaggctgaggagacagtc | | | |
| | gctgccaaggttgagtaa | | | |
| | atggctctgcaagtctgggagactctcaaggaggccatt | 23 | MALQVWETLKEAIVAYTG | 2 |
| | gtcgcttacaccggtctgtctcccgctaccttcttcact | | LSPATFFTVLALGLAVYY | |
| | gtgctcgctcttggtttggccgtctactacgtcatttcg | | VISGFFGTSDYGSHSRDF | |
| | ggcttcttcggcacctcggactacggctctcactctcga | | GEQMQPLPPPVQVGEISE | |
| | gatttcggtgagcagatgcagcccctgcctcctcccgtc | | EELKQYDGSDSKKPLLMA | |
| | caggttggtgagatctctgaggaggagctcaagcagtac | | IKGQIYDVSQSRMFYGPG | |
| | gacggcagtgactccaagaagcctcttctcatggccatc | | GPYALFAGKDASRALAKM | |
| | aagggccagatttacgatgtcagccagagccgaatgttc | | SFEEKDLTGDISGLGPFE | |
| | tacggacccggtggcccttacgctctgtttgccggcaag | | LEALQDWEYKFMSKYVKV | |
| | gatgcttctcgagctctggccaaaatgtcatttgaagag | | GTVEKPVSETDAAPAGES | |
| | aaggacctgactggtgacatttccggactcggcccattc | | AESTNAEVSKPAEDGPSE | |
| | gagcttgaggccctccaggattgggaatacaagttcatg | | SQADKAEETVAAKQKKEK | |
| | tccaagtacgttaaggtcggcaccgttgagaagcccgtc | | KKGRRRVEGVLMEIRGEA | |
| | agcgagacagacgctgctcccgccggtgagtctgctgag | | VGGRKGEGMSREIGRGGG | |
| | tccaccaacgccgaggtgtccaagcccgctgaggacggt | | GADDGNLKGGGIDHGERE | |
| | ccttccgagtcccaggccgacaaggctgaggagacagtc | | RERERESGMS | |
| | gctgccaagcagaagaaggagaagaagaagggccgacga | | | |
| | cgagtcgagggtgttctcatggagatccgaggcgaggcc | | | |
| | gttggtggccgaaagggcgagggcatgtcgagggagatt | | | |
| | ggccgaggtggtggtggtgccgacgacggcaacctcaag | | | |
| | ggtggtggtatcgaccacggtgagcgagaacgagagcga | | | |
| | gaacgagagtccggcatgtcttaa | | | |
| | atggagctgtactggaaggccatggagcagatcgcctgg | 24 | MELYWKAMEQIAWYTGLS | 3 |
| | tacactggtctgtcccccaccgccttcttcaccattctg | | PTAFFTILASMIFVFQMV | |
| | gcctccatgatctttgtgttccagatggtcagctccatg | | SSMFVSPEEFNKPPTVPV | |
| | ttcgtttctcccgaggagttcaacaagcctcccaccgtc | | SSSNPANSNLFVNDSVAD | |
| | cccgtcagctcttccaaccccgccaactccaacctcttc | | ASQAVQIGQLTEQQLRAY | |
| | gtcaacgactctgtggctgatgcttcccaggccgtccag | | DGSDPNKPLLMAIKGQIY | |
| | attggtcagctcaccgagcagcagctccgtgcttatgac | | DVSSGRMFYGPGSPYAMF | |
| | ggcagtgaccccaacaagcctcttctcatggccatcaag | | AGKDASRALALLSFKPED | |
| | ggccagatctacgacgtttcttctggacgaatgttctac | | ITGNIEGLSEEELVILQD | |
| | ggaccgggctctccttacgctatgtttgccggcaaggat | | WEYKFMEKYVKVGELVLE | |
| | gcttctcgagctctggctctgctgtccttcaagcccgag | | GGGEGMNEHSENGHRNFE | |
| | gacatcaccggtaacatcgagggtttgtccgaggaggag | | IDQEEERIEAR | |
| | ctcgttatccttcaggactgggagtacaagttcatggag | | | |
| | aagtacgtcaaggttggtgagcttgtccttgagggagga | | | |
| | ggagaagggatgaacgagcactccgagaacggtcaccga | | | |
| | aactttgagattgaccaggaggaggagcgaatcgaggct | | | |
| | cgataa | | | |
| 934903 | ATGGCTCTGCAAGTCTGGGAGACTCTCAAGGAGGCC | 316 | MALQVWETLKEAIVAYT | 318 |
| | ATTGTCGCTTACACCGGTCTGTCTCCCGCTACCTTC | | GLSPATFFTVLALGLAV | |
| | TTCACTGTGCTCGCTCTTGGTTTGGCCGTCTACTAC | | YYVISGFFGTSDYGSHS | |
| | GTCATTTCGGGCTTCTTCGGCACCTCGGACTACGGC | | RDFGEQMQPLPPPVQVG | |
| | TCTCACTCTCGAGATTTCGGTGAGCAGATGCAGCCC | | EISEEELKQYDGSDSKK | |
| | CTGCCTCCTCCCGTCCAGGTTGGTGAGATCTCTGAG | | PLLMAIKGQIYDVSQSR | |
| | GAGGAGCTCAAGCAGTACGACGGCAGTGACTCCAAG | | MFYGPGGPYALFAGKDA | |
| | AAGCCTCTTCTCATGGCCATCAAGGGCCAGATTTAC | | SRALAKMSFEEKDLTGD | |
| | GATGTCAGCCAGAGCCGAATGTTCTACGGACCCGGT | | ISGLGPFELEALQDWEY | |
| | GGCCCTTACGCTCTGTTTGCCGGCAAGGATGCTTCT | | KFMSKYVKVGTVEKPVS | |
| | CGAGCTCTGGCCAAAATGTCATTTGAAGAGAAGGAC | | ETVAAKVE | |
| | CTGACTGGTGACATTTCCGGACTCGGCCCATTCGAG | | | |
| | CTTGAGGCCCTCCAGGATTGGGAATACAAGTTCATG | | | |
| | TCCAAGTACGTTAAGGTCGGCACCGTTGAGAAGCCC | | | |
| | GTCAGCGAGACAGTCGCTGCCAAGGTTGAGTAG | | | |
| 994375 | ATGGCTCTGCAAGTCTGGGAGACTCTCAAGGAGGCC | 317 | MALQVWETLKEAIVAYT | 1 |
| | ATTGTCGCTTACACCGGTCTGTCTCCCGCTACCTTC | | GLSPATFFTVLALGLAV | |
| | TTCACTGTGCTCGCTCTTGGTTTGGCCGTCTACTAC | | YYVISGFFGTSDYGSHS | |
| | GTCATTTCGGGCTTCTTCGGCACCTCGGACTACGGC | | RDFGEQMQPLPPPVQVG | |
| | TCTCACTCTCGAGATTTCGGTGAGCAGATGCAGCCC | | EISEEELKQYDGSDSKK | |
| | CTGCCTCCTCCCGTCCAGGTTGGTGAGATCTCTGAG | | PLLMAIKGQIYDVSQSR | |
| | GAGGAGCTCAAGCAGTACGACGGCAGTGACTCCAAG | | MFYGPGGPYALFAGKDA | |
| | AAGCCTCTTCTCATGGCCATCAAGGGCCAGATTTAC | | SRALAKMSFEEKDLTGD | |
| | GATGTCAGCCAGAGCCGAATGTTCTACGGACCCGGT | | ISGLGPFELEALQDWEY | |
| | GGCCCTTACGCTCTGTTTGCCGGCAAGGATGCTTCT | | KFMSKYVKVGTVEKPVS | |
| | CGAGCTCTGGCCAAAATGTCATTTGAAGAGAAGGAC | | ETDAAPAGESAESTNAE | |
| | CTGACTGGTGACATTTCCGGACTCGGCCCATTCGAG | | VSKPAEDGPSESQADKA | |
| | CTTGAGGCCCTCCAGGATTGGGAATACAAGTTCATG | | EETVAAKVE | |
| | TCCAAGTACGTTAAGGTCGGCACCGTTGAGAAGCCC | | | |
| | GTCAGCGAGACAGACGCTGCTCCCGCCGGTGAGTCT | | | |
| | GCTGAGTCCACCAACGCCGAGGTGTCCAAGCCCGCT | | | |
| | GAGGACGGTCCTTCCGAGTCCCAGGCCGACAAGGCT | | | |
| | GAGGAGACAGTCGCTGCCAAGGTTGAGTAG | | | |
| 1338488 | ATGGCTCTGCAAGTCTGGGAGACTCTCAAGGAGGCC | 317 | MALQVWETLKEAIVAYT | 1 |
| | ATTGTCGCTTACACCGGTCTGTCTCCCGCTACCTTC | | GLSPATFFTVLALGLAV | |
| | TTCACTGTGCTCGCTCTTGGTTTGGCCGTCTACTAC | | YYVISGFFGTSDYGSHS | |
| | GTCATTTCGGGCTTCTTCGGCACCTCGGACTACGGC | | RDFGEQMQPLPPPVQVG | |
| | TCTCACTCTCGAGATTTCGGTGAGCAGATGCAGCCC | | EISEEELKQYDGSDSKK | |
| | CTGCCTCCTCCCGTCCAGGTTGGTGAGATCTCTGAG | | PLLMAIKGQIYDVSQSR | |
| | GAGGAGCTCAAGCAGTACGACGGCAGTGACTCCAAG | | MFYGPGGPYALFAGKDA | |
| | AAGCCTCTTCTCATGGCCATCAAGGGCCAGATTTAC | | SRALAKMSFEEKDLTGD | |
| | GATGTCAGCCAGAGCCGAATGTTCTACGGACCCGGT | | ISGLGPFELEALQDWEY | |
| | GGCCCTTACGCTCTGTTTGCCGGCAAGGATGCTTCT | | KFMSKYVKVGTVEKPVS | |
| | CGAGCTCTGGCCAAAATGTCATTTGAAGAGAAGGAC | | ETDAAPAGESAESTNAE | |
| | CTGACTGGTGACATTTCCGGACTCGGCCCATTCGAG | | VSKPAEDGPSESQADKA | |
| | CTTGAGGCCCTCCAGGATTGGGAATACAAGTTCATG | | EETVAAKVE | |
| | TCCAAGTACGTTAAGGTCGGCACCGTTGAGAAGCCC | | | |
| | GTCAGCGAGACAGACGCTGCTCCCGCCGGTGAGTCT | | | |
| | GCTGAGTCCACCAACGCCGAGGTGTCCAAGCCCGCT | | | |
| | GAGGACGGTCCTTCCGAGTCCCAGGCCGACAAGGCT | | | |
| | GAGGAGACAGTCGCTGCCAAGGTTGAGTAG | | | |
| 1338489 | ATGGAGCTGTACTGGAAGGCCATGGAGCAGATCGCC | 330 | MELYWKAMEQIAWYTGLS | 3 |
| | TGGTACACTGGTCTGTCCCCCACCGCCTTCTTCACC | | PTAFFTILASMIFVFQMV | |
| | ATTCTGGCCTCCATGATCTTTGTGTTCCAGATGGTC | | SSMFVSPEEFNKPPTVPV | |
| | AGCTCCATGTTCGTTTCTCCCGAGGAGTTCAACAAG | | SSSNPANSNLFVNDSVAD | |
| | CCTCCCACCGTCCCCGTCAGCTCTTCCAACCCCGCC | | ASQAVQIGQLTEQQLRAY | |
| | AACTCCAACCTCTTCGTCAACGACTCTGTGGCTGAT | | DGSDPNKPLLMAIKGQIY | |
| | GCTTCCCAGGCCGTCCAGATTGGTCAGCTCACCGAG | | DVSSGRMFYGPGSPYAMF | |
| | CAGCAGCTCCGTGCTTATGACGGCAGTGACCCCAAC | | AGKDASRALALLSFKPED | |
| | AAGCCTCTTCTCATGGCCATCAAGGGCCAGATCTAC | | ITGNIEGLSEEELVILQD | |
| | GACGTTTCTTCTGGACGAATGTTCTACGGACCGGGC | | WEYKFMEKYVKVGELVLE | |
| | TCTCCTTACGCTATGTTTGCCGGCAAGGATGCTTCT | | GGGEGMNEHSENGHRNFE | |
| | CGAGCTCTGGCTCTGCTGTCCTTCAAGCCCGAGGAC | | IDQEEERIEAR | |
| | ATCACCGGTAACATCGAGGGTTTGTCCGAGGAGGAG | | | |
| | CTCGTTATCCTTCAGGACTGGGAGTACAAGTTCATG | | | |
| | GAGAAGTACGTCAAGGTTGGTGAGCTTGTCCTTGAG | | | |
| | GGAGGAGGAGAAGGGATGAACGAGCACTCCGAGAAC | | | |
| | GGTCACCGAAACTTTGAGATTGACCAGGAGGAGGAG | | | |
| | CGAATCGAGGCTCGATAG | | | |
| 1338490 | ATGGCTCTGCAAGTCTGGGAGACTCTCAAGGAGGCC | 331 | MALQVWETLKEAIVAYTG | 2 |
| | ATTGTCGCTTACACCGGTCTGTCTCCCGCTACCTTC | | LSPATFFTVLALGLAVYY | |
| | TTCACTGTGCTCGCTCTTGGTTTGGCCGTCTACTAC | | VISGFFGTSDYGSHSRDF | |
| | GTCATTTCGGGCTTCTTCGGCACCTCGGACTACGGC | | GEQMQPLPPPVQVGEISE | |
| | TCTCACTCTCGAGATTTCGGTGAGCAGATGCAGCCC | | EELKQYDGSDSKKPLLMA | |
| | CTGCCTCCTCCCGTCCAGGTTGGTGAGATCTCTGAG | | IKGQIYDVSQSRMFYGPG | |
| | GAGGAGCTCAAGCAGTACGACGGCAGTGACTCCAAG | | GPYALFAGKDASRALAKM | |
| | AAGCCTCTTCTCATGGCCATCAAGGGCCAGATTTAC | | SFEEKDLIGDISGLGPFE | |
| | GATGTCAGCCAGAGCCGAATGTTCTACGGACCCGGT | | LEALQDWEYKFMSKYVKV | |
| | GGCCCTTACGCTCTGTTTGCCGGCAAGGATGCTTCT | | GTVEKPVSETDAAPAGES | |
| | CGAGCTCTGGCCAAAATGTCATTTGAAGAGAAGGAC | | AESTNAEVSKPAEDGPSE | |
| | CTGACTGGTGACATTTCCGGACTCGGCCCATTCGAG | | SQADKAEETVAAKQKKEK | |
| | CTTGAGGCCCTCCAGGATTGGGAATACAAGTTCATG | | KKGRRRVEGVLMEIRGEA | |
| | TCCAAGTACGTTAAGGTCGGCACCGTTGAGAAGCCC | | VGGRKGEGMSREIGRGGG | |
| | GTCAGCGAGACAGACGCTGCTCCCGCCGGTGAGTCT | | GADDGNLKGGGIDHGERE | |
| | GCTGAGTCCACCAACGCCGAGGTGTCCAAGCCCGCT | | RERERESGMS | |
| | GAGGACGGTCCTTCCGAGTCCCAGGCCGACAAGGCT | | | |
| | GAGGAGACAGTCGCTGCCAAGCAGAAGAAGGAGAAG | | | |
| | AAGAAGGGCCGACGACGAGTCGAGGGTGTTCTCATG | | | |
| | GAGATCCGAGGCGAGGCCGTTGGTGGCCGAAAGGGC | | | |
| | GAGGGCATGTCGAGGGAGATTGGCCGAGGTGGTGGT | | | |
| | GGTGCCGACGACGGCAACCTCAAGGGTGGTGGTATC | | | |
| | GACCACGGTGAGCGAGAACGAGAGCGAGAACGAGAG | | | |
| | TCCGGCATGTCTTAG | | | |

TABLE 6
Non-Limiting Examples of CDSs.

| | Nucleic acid | Protein |
|---|---|---|
| Name | SEQ ID NO | SEQ ID NO |
| A0A0K9RW03_m | 184 | 224 |
| AquAgaCDS1_m | 185 | 225 |
| AquAgaCDS16 | 186 | 226 |
| AquAgaCDS6 | 187 | 227 |
| BenHisCDS2_m | 188 | 228 |
| A0A0D3QY32 | 189 | 229 |
| A0A0D3QXV2 | 190 | 230 |
| CmaCh17G013880.1 | 191 | 231 |
| A0A1S3CBF6 | 192 | 232 |
| CocGraCDS4 | 193 | 233 |
| CocGraCDS6_m | 194 | 234 |
| CSPI06G07180.1 | 195 | 235 |
| CucFoeCDS | 196 | 236 |
| CucMelMakCDS5 | 197 | 237 |
| CucMetCDS | 198 | 238 |
| CucPepOviCDS1_m | 199 | 239 |
| CucPepOviCDS2 | 200 | 240 |
| CucPepOviCDS3 | 201 | 241 |
| CucPepOviCDS3_m | 202 | 242 |
| Cucsa.349060.1 | 203 | 243 |
| F6GYI4 | 204 | 244 |
| GynCarCDS1 | 205 | 245 |
| GynCarCDS4 | 206 | 246 |
| K7NBZ9 | 207 | 247 |
| LagSicCDS2_m | 208 | 248 |
| Lus10014538.g_m | 209 | 249 |
| Lus10032146.g_m | 210 | 250 |
| MomChaCDS2 | 211 | 251 |
| MomChaCDS4 | 212 | 252 |
| O23909_PEA_Y118L | 213 | 253 |
| Q6BE24 | 214 | 254 |
| SecEduCDS | 215 | 255 |
| SgCDS1 | 216 | 256 |
| SgCDS_Scer1 | 217 | 257 |
| TriKirCDS10 | 218 | 258 |
| TriKirCDS4 | 219 | 259 |
| XP_006340479.1 | 220 | 260 |
| XP_008655662.1 | 221 | 261 |
| XP_010541955.1_m | 222 | 262 |
| XP_016688836.1_m | 223 | 263 |

TABLE 7
Non-Limiting Examples of C11 Hydroxylases (P450s),
Cytochrome P450 Reductases, Epoxide Hydrolases
(EPHs), and Squalene Epoxidases.

| | Nucleic acid | Protein |
|---|---|---|
| Enzyme | SEQ ID NO | SEQ ID NO |
| C11 hydroxylase | 264 | 280 |
| C11 hydroxylase (cucurbitadienol oxidase) | 265 | 281 |
| Cytochrome P450 reductase | 266 | 282 |
| Cytochrome P450 reductase | 267 | 283 |
| Epoxide hydrolase | 268 | 284 |
| Epoxide hydrolase | 269 | 285 |
| Epoxide hydrolase (epoxide hydratase) | 270 | 286 |
| Epoxide hydrolase (epoxide hydratase) | 271 | 287 |
| Epoxide hydrolase (epoxide hydratase) | 272 | 288 |
| Epoxide hydrolase (epoxide hydratase) | 273 | 289 |
| Epoxide hydrolase (epoxide hydratase) | 274 | 290 |
| Epoxide hydrolase (epoxide hydratase) | 275 | 291 |
| Epoxide hydrolase (epoxide hydratase) | 276 | 292 |
| Squalene epoxidase | 277 | 293 |
| Squalene epoxidase | 278 | 294 |
| Squalene epoxidase | 279 | 295 |

TABLE 8
Sequences of Additional Enzymes Associated with the Disclosure

| | Nucleic Acid | Protein |
|---|---|---|
| Name | SEQ ID NO | SEQ ID NO |
| CYP1798 | 296 | 305 |
| AtCPR | 297 | 306 |
| CPR4497 | 298 | 307 |
| sgCDS | 299 | 308 |
| EPH3 | 300 | 309 |
| atEPH2 | 301 | 310 |
| ERG9 | 302 | 311 |
| ERG1 | 303 | 312 |
| ERG7 | 304 | 313 |
| CYP5491 | 314 | 315 |
| SgCDS | 319 | 247 |
| CYP1798 | 320 | 280 |
| CYP5491 -T351M | 321 | 324 |
| SgEPH3 | 322 | 286 |
| AtCPR | 323 | 283 |
| ERG1 | 326 | 328 |
| ERG7 (comprising L491Q, Y586F, and | 327 | 329 |
| R660H relative to SEQ ID NO: 337) | | |
| ERG7 (comprising K47E, L92I, T360S, | 332 | 336 |
| S372P, T444M, and R578P relative to | | |
| SEQ ID NO: 337) | | |
| CPR4497 | 333 | 282 |
| CYP1798 | 334 | 280 |
| CYP1798 | 320 | 280 |
| CYP1798 | 335 | 280 |
| ERG7 | 338 | 337 |

### EQUIVALENTS

Those skilled in the art will recognize, or be able to ascertain using no more than routine experimentation, many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

1. A host cell that comprises a heterologous polynucleotide encoding a cytochrome b5 (CB5), wherein the host cell is capable of producing more mogrol than a control host cell that 5 does not comprise the heterologous polynucleotide, and wherein the CB5 comprises:
a) the amino acid sequence
(SEQ ID NO: 47)
YTGLSP;
b) the amino acid sequence
(SEQ ID NO: 48)
KPLLMAIKGQIYDVS;
c) the amino acid sequence
(SEQ ID NO: 49)
LQDWEYKFM;
and/or
d) the amino acid sequence
(SEQ ID NO: 53)
X₁X₂X₃EX₄GX₅X₆X₇X₈X₉X₁₀D,
wherein:
(i) X₁ is the amino acid K or E;
(ii) X₂ is the amino acid P or H;
(iii) X₃ is the amino acid A or S;
(iv) X₄ is the amino acid D or N;
(v) X₅ is the amino acid P or H;
(vi) X₆ is the amino acid S or R;
(vii) X₇ is the amino acid E or N;
(viii) X₈ is the amino acid S or F;
(ix) X₉ is the amino acid Q or E; and/or
(x) X₁₀ is the amino acid A or I.

2. The host cell of claim 1, wherein the CB5 comprises:
a) the amino acid sequence
X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), wherein:
(i) X₁ is the amino acid E or Q;
(ii) X₂ is the amino acid L or V;
(iii) X₃ is the amino acid Y or W;
(iv) X₄ is the amino acid W or E;
(v) X₅ is the amino acid K or T;
(vi) X₆ is the amino acid A or L;
(vii) X₇ is the amino acid M or K;
(viii) X₈ is the amino acid Q or A;
(ix) X₉ is the amino acid A or V;
(x) X₁₀ is the amino acid W or A;
(xi) X₁₁ is the amino acid T or A;
(xii) X₁₂ is the amino acid A or T;
(xiii) X₁₃ is the amino acid I or V;
(xiv) X₁₄ is the amino acid S or L;
(xv) X₁₅ is the amino acid M or G;
(xvi) X₁₆ is the amino acid I or L;
(xvii) X₁₇ is the amino acid F or A;
(xviii) X₁₈ is the amino acid F or Y;
(xix) X₁₉ is the amino acid Q or Y;
(xx) X₂₀ is the amino acid M or V;
(xxi) X₂₁ is the amino acid V or I;
(xxii) X₂₂ is the amino acid S or G;
(xxiii) X₂₃ is the amino acid M or F;
(xxiv) X₂₄ is the amino acid V or G;
(xxv) X₂₅ is the amino acid S or T;
(xxvi) X₂₆ is the amino acid P or S;
(xxvii) X_{2 7} is the amino acid E or D;
(xxviii) X₂₈ is the amino acid E or Y;
(xxix) X₂₉ is the amino acid F or G;
(xxx) X₃₀ is the amino acid N or S; and/or
(xxi) X₃₁ is the amino acid K or H;
b) the amino acid sequence
X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein:
(i) X₁ is the amino acid P or A;
(ii) X₂ is the amino acid V or I;
(iii) X₃ is the amino acid E or Q;
(iv) X₄ is the amino acid I or L;
(v) X₅ is the amino acid S or T;
(vi) X₆ is the amino acid E or Q;
(vii) X₇ is the amino acid E or Q;
(viii) X₈ is the amino acid K or R;
(ix) X₉ is the amino acid Q or A;
(x) X₁₀ is the amino acid S or P;
(xi) X₁₁ is the amino acid K or N;
(xii) X₁₂ is the amino acid Q or S; and/or (xiii) X₁₃ is the amino acid S or G; and/or
c) the amino acid sequence
LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), wherein:
(i) X₁ is the amino acid K or L;
(ii) X₂ is the amino acid M or L;
(iii) X₃ is the amino acid E or K;
(iv) X₄ is the amino acid E or P;
(v) X₅ is the amino acid K or E;
(vi) X₆ is the amino acid L or I;
(vii) X₇ is the amino acid D or N;
(viii) X₈ is the amino acid S or E;
(ix) X₉ is the amino acid G or S;
(x) X₁₀ is the amino acid P or E;
(xi) X₁₁ is the amino acid F or E;
(xii) X₁₂ is the amino acid E or V;
(xiii) X₁₃ is the amino acid A or I;
(xiv) X₁₄ is the amino acid S or E;
(xv) X₁₅ is the amino acid T or E; and/or
(xvi) X₁₆ is the amino acid V or L.

3. The host cell of claim 1 or 2, wherein the CB5 comprises one or more of the following amino acid sequences:
a)
(SEQ ID NO: 58)
QVWETLKEAIVAYTGLSPATFFTVLA
LGLAVYYVISGFFGTSDYGSH
or
(SEQ ID NO: 59)
ELYWKAMEQIAWYTGLSPTAFFTILA
SMIFVFQMVSSMFVSPEEFNK;
b)
(SEQ ID NO: 60)
PVQVGEISEEELKQYDGSDSKKPLLM
AIKGQIYDVSQSRMF
or
(SEQ ID NO: 61)
AVQIGQLTEQQLRAYDGSDPNKPLLM
AIKGQIYDVSSGRMF;
c)
(SEQ ID NO: 62)
LAKMSFEEKDLTGDISGLGPFELEA
LQDWEYKFMSKYVKVGTV
or
(SEQ ID NO: 63)
LALLSFKPEDITGNIEGLSEEELVIL
QDWEYKFMEKYVKVGEL;
and
d)
(SEQ ID NO: 64)
KPAEDGPSESQAD
or
(SEQ ID NO: 65)
EHSENGHRNFEID

4. The host cell of claim 1, wherein the CB5 comprises:
a) the amino acid sequence YTGLSP (SEQ ID NO: 47) at residues corresponding to positions 16-21 in SEQ ID NO: 1;
b) the amino acid sequence KPLLMAIKGQIYDVS (SEQ ID NO: 48) at residues corresponding to positions 85-99 in SEQ ID NO: 1; and/or
c) the amino acid sequence LQDWEYKFM (SEQ ID NO: 49) at residues corresponding to positions 148-156 in SEQ ID NO: 1

5. The host cell of claim 1 or 3, wherein the CB5 comprises the amino acid sequence X₁X₂X₃EX₄GX₅X₆X₇X₈X₉X₁₀D (SEQ ID NO: 53) at residues corresponding to positions 190-202 of SEQ ID NO: 1.

6. The host cell of claim 2 or claim 3, wherein the CB5 comprises:
a) the amino acid sequence
X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁(SEQ ID NO: 50) at residues corresponding to positions 4-50 of SEQ ID NO: 1;
b) the amino acid sequence
X₁VQX₂GX₃X₄X₅EX₆X₇LX₈X₉YDGSDX₁₀X₁₁KPLLMAIKGQIYDVSX₁₂X₁₃RMF (SEQ ID NO: 51) at residues corresponding to positions 64-104 of SEQ ID NO: 1; and/or
c) the amino acid sequence
LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52) at residues corresponding to positions 123-165 of SEQ ID NO: 1.

7. The host cell of any one of claims 1-6, wherein the CB5 comprises at most one histidine in one or more of the following regions:
a) a region corresponding to positions 64-104 of SEQ ID NO: 1;
b) a region corresponding to positions 105-122 of SEQ ID NO: 1; and/or
c) a region corresponding to positions 123-165 of SEQ ID NO: 1.

8. The host cell of claim 7, wherein the CB5 comprises no histidine residues in:
a) a region corresponding to positions 64-104 of SEQ ID NO: 1;
b) a region corresponding to positions 105-122 of SEQ ID NO: 1; and/or
c) a region corresponding to positions 123-165 of SEQ ID NO: 1.

9. The host cell of any one of claims 1-8, wherein the CB5 comprises a sequence that is at least 90% identical to any one of SEQ ID NOs: 1-3 and 318.

10. The host cell of claim 9, wherein the CB5 comprises the sequence of any one of SEQ ID NOs: 1-3 and 318.

11. The host cell of any one of claims 1-10, wherein the heterologous polynucleotide comprises a sequence that is at least 90% identical to any one of SEQ ID NOs: 11-14, 22-24, 316-317, and 330-331.

12. The host cell of claim 11, wherein the heterologous polynucleotide comprises the sequence of any one of SEQ ID NOs: 11-14, 22-24, 316-317, and 330-331.

13. A host cell that comprises a heterologous polynucleotide encoding a cytochrome b5 (CB5), wherein the CB5 comprises a sequence that is at least 90% identical to any one of SEQ ID NOs: 1-10 and 318 and wherein the host cell is capable of producing mogrol.

14. The host cell of claim 13, wherein the CB5 comprises the sequence of any one of SEQ ID NOs: 1-10 and 318.

15. A host cell that comprises a heterologous polynucleotide encoding a cytochrome b5 (CB5), wherein the CB5 comprises a sequence that is at least 90% identical to any one of SEQ ID NOs: 1-4 and 318 and wherein the host cell is capable of producing more mogrol than a control host cell that does not comprise the heterologous polynucleotide.

16. A host cell that comprises a heterologous polynucleotide encoding a cytochrome b5 (CB5), wherein the heterologous polynucleotide comprises a sequence that is at least 90% identical to any one of SEQ ID NOs: 11-24, 316-317, and 330-331, and wherein the host cell is capable of producing mogrol.

17. The host cell of claim 16, wherein the heterologous polynucleotide comprises the sequence of any one of SEQ ID NOs: 11-24 316-317, and 330-331.

18. A host cell that comprises a heterologous polynucleotide encoding a cytochrome b5 (CB5), wherein the CB5 comprises:
a) the amino acid sequence
(SEQ ID NO: 54)
ILRVSFRKYRKAIEQ;
b) the amino acid sequence
(SEQ ID NO: 55)
RAFRPSIRFKKSHSTVPT;
c) the amino acid sequence
(SEQ ID NO: 56)
KNTLYVGG;
and/or
d) the amino acid sequence
(SEQ ID NO: 57)
DQATQKHRSFGFVTFLEKED
and wherein the host cell is capable of producing more mogrol than a control host cell that does not comprise the heterologous polynucleotide.

19. The host cell of claim 18, wherein the CB5 comprises:
a) the amino acid sequence ILRVSFRKYRKAIEQ (SEQ ID NO: 54) at residues corresponding to positions 23-37 of SEQ ID NO: 4;
b) the amino acid sequence RAFRPSIRFKKSHSTVPT (SEQ ID NO: 55) at residues corresponding to positions 53-70 of SEQ ID NO: 4;
c) the amino acid sequence KNTLYVGG (SEQ ID NO: 56) at residues corresponding to positions 168-175 of SEQ ID NO: 4; and/or
d) the amino acid sequence DQATQKHRSFGFVTFLEKED (SEQ ID NO: 57) at residues corresponding to positions 203-222 of SEQ ID NO: 4.

20. The host cell of claim 18 or 19, wherein the CB5 comprises a sequence that is at least 90% identical to SEQ ID NO: 4.

21. The host cell of claim 20, wherein the CB5 comprises SEQ ID NO: 4.

22. The host cell of any one of claims 18-21, wherein the heterologous polynucleotide comprises a sequence that is at least 90% identical to SEQ ID NO: 15.

23. The host cell of claim 22, wherein the heterologous polynucleotide comprises SEQ ID NO: 15.

24. The host cell of any one of claims 1-23, wherein the host cell is capable of producing more than 13.5 mg/L mogrol.

25. The host cell of any one of claims 1-24, wherein the host cell further comprises one or more heterologous polynucleotides encoding one or more of: a UDP-glycosyltransferases (UGT) enzyme, a cucurbitadienol synthase (CDS) enzyme, a C11 hydroxylase, a cytochrome P450 reductase, an epoxide hydrolase (EPH), a lanosterol synthase, and a squalene epoxidase (SQE).

26. The host cell of claim 25, wherein the UGT enzyme comprises a sequence that is at least 90% identical to SEQ ID NO: 121.

27. The host cell of claim 25 or 26, wherein the CDS enzyme comprises a sequence that is at least 90% identical to any one of SEQ ID NOs: 226, SEQ ID NO: 235, and SEQ ID NO: 232.

28. The host cell of any one of claims 25-27, wherein the C11 hydroxylase comprises a sequence that is at least 90% identical to any one of SEQ ID NOs: 280-281, 305, 315, and 324.

29. The host cell of any one of claims 25-28, wherein the cytochrome P450 reductase comprises a sequence that is at least 90% identical to any one of SEQ ID NOs: 282-283 and 306-307.

30. The host cell of any one of claims 25-29, wherein the EPH comprises a sequence that is at least 90% identical to any one of SEQ ID NOs: 284-292 and 309-310.

31. The host cell of any one of claims 25-30, wherein the SQE comprises a sequence that is at least 90% identical to any one of SEQ ID NOs: 293-295, 312, or 328.

32. The host cell of any one of claims 25-31, wherein the lanosterol synthase comprises a sequence that is at least 90% identical to SEQ ID NO: 329 or 336.

33. The host cell of any one of claims 25-32, wherein the SQE comprises a sequence that is at least 90% identical to SEQ ID NO: 312 or 328.

34. The host cell of any one of claims 1-33, wherein the host cell is a yeast cell, a plant cell, or a bacterial cell.

35. The host cell of claim 34, wherein the host cell is a yeast cell.

36. The host cell of claim 35, wherein the yeast cell is a Saccharomyces cerevisiae.

37. The host cell of claim 35, wherein the yeast cell is a Yarrowia lipolytica cell.

38. The host cell of claim 34, wherein the host cell is a bacterial cell.

39. The host cell of claim 38, wherein the bacterial cell is an E. coli cell.

40. A method of producing mogrol comprising culturing the host cell of any one of claims 1-39.

41. A method of producing a mogroside comprising culturing the host cell of any one of claims 1-39.

42. The method of claim 40 or 41, wherein the mogroside is selected from mogroside I-A1 (MIA1), mogroside IE (MIE), mogroside II-A1 (MIIA1), mogroside II-A2 (MIIA2), mogroside III-A1 (MIIIA1), mogroside II-E (MIIE), mogroside III (MITI), siamenoside I, mogroside IV (MIV), mogroside IVa (MIVA), isomogroside IV, mogroside III-E (MIIIE), mogroside V (MV), and/or mogroside VI (MVI).

43. A bioreactor for producing mogrol or mogrosides, wherein the bioreactor comprises a host cell of any one of claims 1-39.

44. A non-naturally occurring polynucleotide comprising a sequence that is at least 90% identical to any one of SEQ ID NOs: 11-14, 22-24, 316-317, and 330-331.

45. The non-naturally occurring polynucleotide of claim 44, wherein the polynucleotide encodes a cytochrome b5 (CB5) comprising a sequence that is at least 90% identical to any one of SEQ ID NOs: 1-10 and 318.

46. An expression vector comprising the non-naturally occurring polynucleotide of claim 44 or 45.
